(19) United States
(12) Patent Application Publication
(10) Pub. No.: US 2023/0287435 A1
(43) Pub. Date: Sep. 14, 2023

(54) MICROORGANISMS AND METHODS FOR REDUCING BY-PRODUCTS

(71) Applicant: Genomatica, Inc., San Diego, CA (US)

(72) Inventors: Harish Nagarajan, San Diego, CA (US); Gionata Scalcinati, Carlsbad, CA (US); Tae Hoon Yang, Encinitas, CA (US)

(21) Appl. No.: 17/772,990

(22) PCT Filed: Oct. 28, 2020
(86) PCT No.: PCT/US2020/057707

§ 371 Date: Apr. 28, 2022

Related U.S. Application Data
(60) Provisional application No. 62/928,183, filed on Oct. 30, 2019.

(51) Int. Cl. C12N 15/52; C12P 7/18

(52) U.S. Cl. CPC C12N 15/52; C12P 7/18; C12Y 202/01006; C12Y 104/99001; C12Y 501/01001; C12Y 102/01003; C12Y 602/01001; C12Y 203/03001

(57) ABSTRACT

The present disclosure provides microbial organisms having decreased production of unwanted by-products (e.g, pyruvate-, CO₂—, TCA-derived by-products; acetate; ethanol; and/or, alanine) to enhance carbon flux through acetyl-CoA, which can increase production of acetyl-CoA derived compounds (e.g, 1,3-BDO, MMA, and (3R)-hydroxybutyl (3R)-hydroxybutyrate, or any other acetyl-CoA derived compounds), and products made from any of these compounds. Also provided are one or more exogenous nucleic acids encoding enzymes that can decrease production of unwanted by-products (e.g, aldehyde dehydrogenase, acetyl-CoA synthase, amino acid dehydrogenase, alanine racemase, and/or citrate synthase), and/or one or more gene attenuations occurring in genes (e.g., acetolactate synthase) that result in decreased production of unwanted by-products. Various combinations of the exogenous nucleic acids and gene deletions are also provided in the present disclosure. Methods of making and using the same, including methods for culturing cells, and for the production of the various products are also provided.

### CROSS-REFERENCE TO RELATED APPLICATIONS

This application claims the benefit of U.S. Provisional Patent Application No. 62/928,183, filed Oct. 30, 2019, which is incorporated by reference herein in its entirety.

### 1. FIELD

The present invention relates generally to organisms engineered to produce desired products, engineered enzymes that facilitate production of a desired product, and more specifically to non-naturally occurring organisms that can reduce by-products, such as pyruvate by-products (e.g., alanine, and/or valine), TCA derived by-products, acetate and/or ethanol, for enhancing carbon flux through acetyl-CoA and thereby increasing one or more acetyl-CoA derived product, including but not limited to 1,3-butanediol (1,3-BDO), methyl methacrylate (MMA), (3R)-hydroxybutyl (3R)-hydroxybutyrate, 3-hydroxybutyrate (3-HB), hexamethylenediamine (HMDA), caprolactam, adipate, 6-aminocaproic acid (6-ACA), methacrylic acid (MAA), fatty acid methyl ester (FAME) and related products derived therefrom.

### 2. BACKGROUND

Microbial organisms can be used for the production of acetyl-CoA derived chemical compounds, such as 1,3-BDO, fatty acid methyl esters (e.g., (3R)-hydroxybutyl (3R)-hydroxybutyrate). The titer, rate, and yield of such production can be limited by the generation of unwanted by-products. In particular, the generation of unwanted by-products, such as pyruvate by-products, acetate and/or ethanol, can limit the amount of acetyl-CoA that is available for the generation of the desired acetyl-CoA derived product. For example, the generation of the pyruvate by-product valine can limit the amount of pyruvate that is available for conversion into acetyl-CoA. Similarly, the generation of unwanted ethanol and/or acetate from acetyl-CoA can limit the amount of acetyl-CoA that is available for conversion into a desired acetyl-CoA by-product. Accordingly, decreased production of unwanted by-products, such as pyruvate by-products, acetate and/or ethanol, can help to increase the titer, rate, and yield of acetyl-CoA derived chemical compounds, such as 1,3-BDO, MMA, and (3R)-hydroxybutyl (3R)-hydroxybutyrate.

1,3-BDO is a four carbon diol traditionally produced from acetylene via its hydration. The resulting acetaldehyde is then converted to 3-hydroxybutyraldehyde which is subsequently reduced to form 1,3-BDO. More recently, acetylene has been replaced by the less expensive ethylene as a source of acetaldehyde. 1,3-BDO is commonly used as an organic solvent for food flavoring agents. It is also used as a co-monomer for polyurethane and polyester resins and is widely employed as a hypoglycemic agent. Optically active 1,3-BDO is a useful starting material for the synthesis of biologically active compounds and liquid crystals. Another use of 1,3-butanediol is that its dehydration affords 1,3-butadiene (Ichikawa et al. Journal of Molecular Catalysis A-Chemical 256:106-112 (2006); Ichikawa et al. Journal of Molecular Catalysis A-Chemical 231:181-189 (2005), which is useful in the manufacture synthetic rubbers (e.g., tires), latex, and resins. The reliance on petroleum based feedstocks for either acetylene or ethylene warrants the development of a renewable feedstock based route to 1,3-butanediol and to butadiene.

MMA is an organic compound with the formula CH₂═C(CH₃)CO₂CH₃. This colorless liquid is the methyl ester of methacrylic acid (MAA) and is the monomer for the production of the transparent plastic polymethyl methacrylate (PMMA). The principal application of methyl methacrylate is the production of polymethyl methacrylate acrylic plastics. Also, methyl methacrylate is used for the production of the co-polymer methyl methacrylate-butadiene-styrene (MBS), used as a modifier for PVC. Methyl methacrylate polymers and co-polymers are used for waterborne coatings, such as latex paint. Uses are also found in adhesive formulations. Contemporary applications include the use in plates that keep light spread evenly across liquid crystal display (LCD) computer and TV screens. Methyl methacrylate is also used to prepare corrosion casts of anatomical organs, such as coronary arteries of the heart.

The intake of compounds and compositions containing (R)-3-hydroxybutyrate derivatives, e.g. (3R)-hydroxybutyl (3R)-hydroxybutyrate, have been shown to boost the levels of ketone bodies in the blood. Ketone bodies are chemical compounds which are produced when fatty acids are metabolized by the body for energy, which can in turn lead to the ketone bodies themselves being used for energy. Ketone bodies have been shown as being suitable for reducing the levels of free fatty acids circulating in the plasma of an individual. Ingestion of ketone bodies can also lead to various clinical benefits, including an enhancement of physical and cognitive performance and treatment of cardiovascular conditions, diabetes and treatment of mitochondrial dysfunction disorders and in treating muscle fatigue and impairment. However, direct administration of ketone bodies is impractical and dangerous. For example, direct administration of either (R)-3-hydroxybutyrate can result in significant acidosis following rapid absorption from the gastrointestinal tract. Administration of the sodium salt of these compounds is also unsuitable due to a potentially dangerous sodium overload that would accompany administration of therapeutically relevant amounts of these compounds. Administration of (R)-3-hydroxybutyrate derivatives in oligomeric form has been used to circumvent this problem. To gain desirable therapeutic and other benefits, the ketone body generally needs to be present in the blood plasma of an individual at a threshold level, for example at least 1 mM (3R)-hydroxybutyl (3R)-hydroxybutyrate. However, low yields, or impracticability on a large scale have hindered production.

Thus, there exists a need for the development of methods to decrease the production of unwanted by-products, such as pyruvate by-products, acetate and/or ethanol, for increasing the efficiency and effectively producing commercial quantities of compounds such as 1,3-BDO, MMA, and (3R)-hydroxybutyl (3R)-hydroxybutyrate. The present invention satisfies these needs and provides related advantages as well. Additional product molecules that can be produced by the teachings of this invention include any acetyl-CoA derived product, including but not limited to adipate, caprolactam, 6-aminocaproic acid (6-ACA), hexametheylenediamine (HMDA), or methacrylic acid (MAA).

### 3. SUMMARY OF INVENTION

In one aspect, provided herein is a non-naturally occurring microbial organism having reduced by-products, includes a microbial organism having a glycolysis pathway and an enhanced carbon flux through acetyl-CoA, the microbial organism comprising one or more of: (a) an attenuated acetolactate synthase; and (b) an acetaldehyde recycling loop.

In some embodiments, the attenuated acetolactate synthase comprises a deletion of acetolactate synthase. In some embodiments, the attenuated acetolactate synthase comprises a non-functional acetolactate synthase. In some embodiments, the acetolactate synthase is ilvG. In some embodiments, the attenuated acetolactate synthase decreases the biosynthesis of valine.

In some embodiments, the acetaldehyde recycling loop comprises at least one exogenous nucleic acid encoding an acetaldehyde recycling loop enzyme selected from the group consisting of an aldehyde dehydrogenase and an acetyl-CoA synthase. In some embodiments, the aldehyde dehydrogenase is AldB. In some embodiments, the acetyl-CoA synthase is an acetyl-CoA synthase variant. In some embodiments, the at least one exogenous nucleic acid is a heterologous nucleic acid.

In certain embodiments, the non-naturally occurring microbial organism of the present disclosure has reduced acetate, ethanol, or a combination thereof.

In some embodiments, the non-naturally occurring microbial organism is in a substantially anaerobic culture medium.

In some embodiments, the non-naturally occurring microbial organism of the present disclosure includes an 1,3-butanediol (1,3-BDO) pathway, an (3R)-hydroxybutyl (3R)-hydroxybutyrate pathway, a 3-hydroxybutyryl-coenzyme A (3HB-CoA) pathway, a methyl methacrylate (MMA) pathway, an adipate pathway, a caprolactam pathway, a 6-aminocaproic acid (6-ACA) pathway, a hexametheylenediamine (HMDA) pathway, or a methacrylic acid (MAA) pathway.

In some embodiments, the microbial organism comprises a 1,3-BDO pathway. In specific embodiments, the 1,3-BDO pathway comprises a thiolase; an acetoacetyl-CoA reductase (CoA-dependent, aldehyde forming); a 3-oxobutyraldehyde reductase (ketone reducing); a 3-hydroxybutyraldehyde reductase; an acetoacetyl-CoA reductase (CoA-dependent, alcohol forming); a 3-oxobutyraldehyde reductase (aldehyde reducing); a 4-hydroxy, 2-butanone reductase; an acetoacetyl-CoA reductase (ketone reducing); a 3-hydroxybutyryl-CoA reductase (aldehyde forming); and a 3-hydroxybutyryl-CoA reductase (alcohol forming).

In some embodiments, the microbial organism comprises an (3R)-hydroxybutyl (3R)-hydroxybutyrate pathway. In specific embodiments, the (3R)-hydroxybutyl (3R)-hydroxybutyrate pathway comprises a thiolase; a (3R)-hydroxybutyl (3R)-hydroxybutyrate ester forming enzyme; a (3R)-hydroxybutyryl-CoA:(R)-1,3-butanediol alcohol transferase; a (3R)hydroxybutyl 3-oxobutyrate ester forming enzyme; an acetoacetyl-CoA:(R)-1,3-butanediol alcohol transferase; a (3R)-hydroxybutyl 3-oxobutyrate reductase; a (3R)-hydroxybutyryl-ACP:(R)-1,3-butanediol ester synthase, and an acetoacetyl-ACP:(R)-1,3-butanediol ester synthase.

In some embodiments, the microbial organism comprises a 3HB-CoA pathway. In specific embodiments, the 3HB-CoA pathway comprises an acetyl-CoA thiolase, and a 3-hydroxybutyryl-CoA dehydrogenase.

In some embodiments, the microbial organism comprises a MMA pathway. In specific embodiments, the MMA pathway comprises: (a) a 4-hydroxybutyryl-CoA dehydratase, a crotonase, a 2-hydroxyisobutyryl-CoA mutase, a 2-hydroxyisobutyryl-CoA dehydratase, and a methacrylic acid (MAA)-CoA: methanol transferase; or (b) a 4-hydroxybutyryl-CoA dehydratase, a crotonase, a 2-hydroxyisobutyryl-CoA mutase, a 3-hydroxyisobutyryl-CoA: methanol transferase, and a methyl-2-hydroxyisobutyrate dehydratase. In some embodiments, the non-naturally occurring microbial organism having a MMA pathway further includes a second MMA pathway comprising: (c) a methacrylic acid (MAA)-CoA: methanol transferase, a 4-hydroxybutyryl-CoA mutase, and a 3-hydroxyisobutyryl-CoA dehydratase; or (d) a 4-hydroxybutyryl-CoA mutase, a 3-hydroxyisobutyryl-CoA: methanol transferase, and a methyl-3-hydroxyisobutyrate dehydratase.

In some embodiments, the microbial organism comprises a 6-ACA pathway. In specific embodiments, the 6-ACA pathway comprises a 2-amino-7-oxosubarate keto-acid decarboxylase, a 2-amino-7-oxoheptanoate decarboxylase, a 2-amino-7-oxoheptanoate oxidoreductase, a 2-aminopimelate decarboxylase, a 6-aminohexanal oxidoreductase, a 2-amino-7-oxoheptanoate decarboxylase, or a 2-amino-7-oxosubarate amino acid decarboxylase.

In some embodiments, the non-naturally occurring microbial organism comprises a caprolactam pathway. In specific embodiments, the caprolactam pathway comprises 3-oxoadipyl-CoA thiolase, 3-oxoadipyl-CoA reductase, 3-hydroxyadipyl-CoA dehydratase, 5-carboxy-2-pentenoyl-CoA reductase, adipyl-CoA reductase (aldehyde forming), 6-aminocaproate transaminase, 6-aminocaproate dehydrogenase, 6-aminocaproyl-CoA/acyl-CoA transferase, and 6-aminocaproyl-CoA synthase.

In some embodiments, the microbial organism comprises an adipate pathway. In specific embodiments, the adipate pathway comprises 3-oxoadipyl-CoA thiolase, 3-oxoadipyl-CoA reductase, 3-hydroxyadipyl-CoA dehydratase, 5-carboxy-2-pentenoyl-CoA reductase, adipyl-CoA hydrolase, adipyl-CoA ligase, adipyl-CoA transferase and phosphotransadipylase/adipate kinase.

In some embodiments, the microbial organism comprises a hexamethylenediamine (HMDA) pathway. In specific embodiments, the HMDA pathway comprise 3-oxoadipyl-CoA thiolase, 3-oxoadipyl-CoA reductase, 3-hydroxyadipyl-CoA dehydratase, 5-carboxy-2-pentenoyl-CoA reductase, adipyl-CoA reductase (aldehyde forming), 6-aminocaproate transaminase, 6-aminocaproate dehydrogenase, 6-aminocaproyl-CoA/acyl-CoA transferase, 6-aminocaproyl-CoA synthase, 6-aminocaproyl-CoA reductase (aldehyde forming), HMDA transaminase, and HMDA dehydrogenase.

In some embodiments, the microbial organism comprises a MAA pathway. In specific embodiments, the MAA pathway comprises: (a) (i) a succinyl-CoA transferase, ligase, or synthetase; (ii) a methylmalonyl-CoA mutase; (iii) a methylmalonyl-CoA epimerase; (iv) a methylmalonyl-CoA reductase (aldehyde forming); (v) a methylmalonate semialdehyde reductase; and (vi) a 3-hydroxyisobutyrate dehydratase; (b) (i) a succinyl-CoA transferase, ligase, or synthetase; (ii) a methylmalonyl-CoA mutase; (iii) a methylmalonyl-CoA reductase (aldehyde forming); (iv) a methylmalonate semialdehyde reductase; and (v) a 3-hydroxyisobutyrate dehydratase; or (c) (i) a succinyl-CoA transferase, ligase, or synthetase; (ii) a methylmalonyl-CoA mutase; (iii) a methylmalonyl-CoA reductase (alcohol forming); and (iv) a 3-hydroxyisobutyrate dehydratase.

In some embodiments, the non-naturally occurring microbial organism provided herein is a species of bacteria, yeast, or fungus.

In another aspect, provided herein is a method for enhancing the carbon flux through acetyl-CoA in a non-naturally occurring microbial organism to increase the yield of an acetyl-CoA derived product, the method comprising culturing the non-naturally occurring microbial organism of the present disclosure under conditions and for a sufficient period of time to produce the acetyl-CoA derived product.

In some embodiments, the acetyl-CoA derived product is selected from a group consisting of 1,3-butanediol (1,3-BDO), methyl methacrylate (MMA), 3R-hydroxybutyric acid-3R-hydroxybutryrate, 3-hydroxybutyrate (3-HB), 4-hydroxy-2-butanone (4OH2B), hexamethylenediamine (HMDA), caprolactam, adipate, 6-aminocaproic acid (6-ACA), and methacrylic acid (MAA).

In some embodiments, the acetyl-CoA derived product comprises 1,3-BDO. In other embodiments, the acetyl-CoA derived product comprises MMA. In further embodiments, the acetyl-CoA derived product comprises 3R-hydroxybutyric acid-3R-hydroxybutryrate.

### 4. BRIEF DESCRIPTION OF THE DRAWINGS

FIG. 1A-FIG. 1C show that the ilvGM deletion strains (L16410 and L16411) performed better than control (L16375) with regard to specific 1,3-BDO production (FIG. 1A), rate (FIG. 1B), and yield [c-mol %] (FIG. 1C), independent from the oxygen transfer rate (OTR).

FIG. 2A-FIG. 2C show that there was an inverse relationship between valine production and 1,3-BDO yield (FIG. 2A) among different bacterial strains. ilvGM deletion strains (L16410 and L16411) had negligible levels of valine (FIG. 2B), and increased levels of 1,3-BDO for each of the culture volumes (FIG. 2C).

FIG. 3 shows an exemplary 1,3-BDO production pathway involving generation of pyruvate by glycolysis, conversion of pyruvate to acetyl-CoA, and acetyl-CoA conversion to 1,3-BDO. The unwanted by-products, ethanol (EtOH) and acetate can be generated from acetyl-CoA by ALD and ADH, or through an acetyl-CoA hydrolase/thioesterase enzyme.

FIG. 4 shows an exemplary 1,3-BDO production pathway that includes an acetaldehyde recycling loop that converts acetaldehyde to acetate by aldB and/or acetate to acetyl-CoA by ACS, which can then be converted to 1,3-BDO.

FIG. 5 shows that specific AldB enzymes have specific activity for acetaldehyde and not 3-HB aldehyde.

FIG. 6A and FIG. 6B shows that expression of ACS* improved the production of 1,3-BDO in the L16946 and L17787 strains, relative to the non-ACS* expressing strains L16768 and L17787, respectively (FIG. 6A). In addition, ACS* expression significantly reduced acetate, but not ethanol formation, indicating that the acetate recycle is efficiently competing with CoA hydrolase (FIG. 6B).

FIG. 7A-FIG. 7D show the that overall product distribution demonstrated that expression of ACS* significantly reduced by-products (FIG. 7C) and increased 1,3-BDO production (FIG. 7D), relative to the non-ACS* expressing strains (FIG. 7A and FIG. 7B).

FIG. 8 shows that co-expression of ACS* and AldB2886B significantly reduced both acetate and ethanol production. Moreover, the overall carbon-2 (i.e., ethanol and acetate) reduction was similar between both strains.

FIG. 9A and FIG. 9B show that strains overexpressing ACS* and the alternative AldB candidate (AldB 1139A), with or without the NadK variant (NadK*), resulted in an increase in 1,3-BDO (FIG. 9A) and a decrease in both ethanol and acetate (FIG. 9B), relative to their respective control strains without ACS* and AldB overexpression.

FIG. 10 shows an exemplary 1,3-BDO production pathway involving generation of pyruvate by glycolysis, and conversion of pyruvate to either acetyl-CoA or L-alanine. An alanine recycling loop involving dadX and dadA converts L-alanine to D-alanine and D-alanine to pyruvate, respectively, thereby decreasing unwanted production of alanine.

FIG. 11A-FIG. 11B show that an alanine recycling loop can decrease the alanine concentration levels across multiple aeration parameters. Microorganisms that expressed dadX and dadA (“recycle”; empty columns) had lower levels of D-Alanine, L-Alanine, total Alanine, and L-Alanine/D-Alanine ratio levels following fermentation (FIG. 11A), relative to control microorganisms without an alanine recycling loop (black columns). Measurement of alanine concentration (mmol/L) throughout fermentation also demonstrated lower levels of alanine in microorganisms with an alanine recycling loop (solid gray line), relative to control microorganisms without an alanine recycling loop (dashed black line) (FIG. 11B).

FIG. 12 shows an exemplary 1,3-BDO production pathway involving the use of acetyl-CoA as a substrate for either conversion into 1,3-BG or conversion into citrate via citrate synthase (gltA). A citrate synthase variant (gltA R109L, “gltA*”) has increased sensitivity to NADH relative to wild-type gltA.

FIG. 13A and FIG. 13B show that microorganisms expressing the citrate synthase variant gltA* (dashed gray line) had a fast microaerobic transition relative to microorganisms expressing the wild-type gltA (solid black line), as indicated by the arrow (FIG. 13A). The microorganisms expressing the citrate synthase variant gltA* (gray column) had increased titers of 1,3-BDO, relative to microorganisms expressing the wild-type gltA (black column) (FIG. 13B).

### 5. DETAILED DESCRIPTION OF THE INVENTION

This invention is directed, in part, to engineered biosynthetic routes to decrease by-products, such as pyruvate by-products (e.g., alanine, and/or valine), tricarboxylic acid cycle (TCA) derived by-products, acetate and/or ethanol, for enhancing carbon flux through acetyl-CoA and thereby increasing acetyl-CoA derived products. Exemplary product molecules include, without limitation, 1,3-butanediol (1,3-BDO), methyl methacrylate (MMA), (3R)-hydroxybutyl (3R)-hydroxybutyrate, 4-hydroxy-2-butanone (4OH2B), 3HB, adipate, caprolactam, 6-ACA, HMDA, or MAA, although given the teachings and guidance provided herein, it will be recognized by one skilled in the art that any product molecule that is derived from acetyl-CoA can exhibit enhanced product production through decreased by-products. The present invention provides non-naturally occurring microbial organisms having one or more exogenous genes encoding enzymes and/or one or more attenuated enzymes that can decrease the production of by-products, such as pyruvate by-products (e.g., alanine, and/or valine), TCA derived by-products, acetate and/or ethanol. In some embodiments, these non-naturally occurring microbial organisms also have one of more exogenous genes encoding enzymes that can catalyze the production of a desired product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In numerous engineered pathways, realization of maximum product yields based on carbohydrate feedstock is hampered by the production of unwanted by-products. In accordance with some embodiments, the present invention increases the yields of acetyl-CoA derived products by (i) decreasing the production of pyruvate by-products, such as valine, to increase the conversion of pyruvate into acetyl-CoA, (ii) recycling unwanted pyruvate by-products, such as alanine, back into pyruvate to increase the availability of pyruvate conversion into acetyl-CoA, (iii) decreasing the entry of acetyl-CoA into the TCA cycle and/or (iv) recycling acetyl-CoA by-products, such as acetate and/or ethanol, back into acetyl-CoA. Products that can be produced by non-naturally occurring organisms and methods described herein include by way of example, but without limitation, 1,3-BDO, fatty acid methyl esters (e.g., MMA), (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins that result in an increase or decrease of a metabolic factor, for example. One exemplary metabolic factor includes acetyl-CoA. Exemplary metabolic factors also include, for example, 1,3-butanediol (1,3-BDO), methyl methacrylate (MMA), and/or 3-hydroxybutyrate (3-HB). Further exemplary metabolic factors include, for example, acetolactate synthase, aldehyde dehydrogenase, and acetyl-CoA synthase.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “by-product” refers to an undesired product produced during the production of a desired product. By way of example, as provided herein, the production of, for example, valine and/or alanine from pyruvate can be considered a by-product where pyruvate is desired to be converted into acetyl-CoA. Similarly, an exemplary by-product includes acetate from acetyl-CoA where acetyl-CoA is desired to be converted into 1,3-BDO, MMA, (3R)-hydroxybutyl (3R)-hydroxybutyrate, or any other acetyl-CoA derived product.

As used herein, the phrases “enhanced carbon flux” or “enhanced carbon flow” are intended to mean to intensify, increase, or further improve the extent or flow of metabolic carbon through or to a desired pathway, pathway product, intermediate, or bioderived compound. The intensity, increase or improvement can be relative to a predetermined baseline of a pathway product, intermediate or bioderived compound. For example, an increased yield of acetyl-CoA can be achieved with an attenuated acetolactate synthase described herein, as compared to with a functional acetolactate synthase. Similarly, an increased yield of acetyl-CoA can be achieved by expressing one or more enzymes of an acetaldehyde recycling loop pathway, as compared to in the absence of an enzyme of an acetaldehyde recycling loop pathway. It is understood that since an increased yield of acetyl-CoA can be achieved, a higher yield of any acetyl-CoA derived compound, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, can also be achieved.

As used herein, the term “recycling loop” refers to one or more reactions that convert a by-product back to a substrate that can then be metabolized and redirected to the desired product. For example, an exemplary recycling loop provided herein includes an acetaldehyde recycling loop which, for example, converts the by-product acetaldehyde to acetyl-CoA to lower acetaldehyde and increase the yield of the desired acetyl-CoA derived product, such as 1,3-BDO, MMA, (3R)-hydroxybutyl (3R)-hydroxybutyrate, or any other acetyl-CoA derived product. Similarly, another exemplary recycling loop includes an alanine-recycling loop which, for example, converts the by-product alanine to pyruvate to lower alanine level and increase the carbon flux from pyruvate to acetyl-CoA, thereby increasing the yield of the desired acetyl-CoA derived product, such as 1,3-BDO, MMA, (3R)-hydroxybutyl (3R)-hydroxybutyrate, or any other acetyl-CoA derived product

As used herein, the term “attenuate,” or grammatical equivalents thereof, is intended to mean to weaken, reduce or diminish the activity or amount of an enzyme or protein. Attenuation of the activity or amount of an enzyme or protein can mimic complete disruption if the attenuation causes the activity or amount to fall below a critical level required for a given pathway, reaction, or series of reactions to function. However, the attenuation of the activity or amount of an enzyme or protein that mimics complete disruption for one pathway, reaction, or series of reactions, can still be sufficient for a separate pathway, reaction, or series of reactions to continue to function. For example, attenuation of an endogenous enzyme or protein can be sufficient to mimic the complete disruption of the same enzyme or protein for production of valine of the invention, but the remaining activity or amount of enzyme or protein can still be sufficient to maintain other pathways, such as a pathway that is critical for the host microbial organism to survive, reproduce or grow. Attenuation of an enzyme or protein can also be weakening, reducing or diminishing the activity or amount of the enzyme or protein in an amount that is sufficient to increase yield of a factor, such as acetyl-CoA derived product, of the invention, but does not necessarily mimic complete disruption of the enzyme or protein.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired enzyme or protein required for a pathway, reaction, or series of reactions. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “variant” is intended to mean a form or version of an enzyme that differs from the wild-type enzyme. An exemplary variant is a mutant version of the enzyme where the amino acid sequence of the variant enzyme differs from the amino acid sequence at one of more at one or more of the homologous amino acids. A variant may have a different function or activity relative to the wild-type enzyme. However, a variant need not be a mutant, and can encompass polymorphisms, paralogs or orthologs.

As used herein, when used in reference to culture or growth conditions, the term “substantially anaerobic” is the amount of oxygen, that is less than about 10% of the saturated amount of dissolved oxygen in the liquid medium it is assumed that the meaning to. This term is maintained in an atmosphere of oxygen of less than about 1%, the sealing chamber of a liquid or solid medium, is also included.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “1,3-butanediol,” or “1,3-BDO” is intended to mean one of four stable isomers of butanediol having the chemical formula C₄H₁₀O₂ and a molecular mass of 90.12 g/mol. The chemical compound 1,3-butanediol is known in the art as 1,3-butylene glycol (1,3-BG) and is also a chemical intermediate or precursor for a family of compounds commonly referred to as the BDO family of compounds.

As used herein, “methyl methacrylate,” or “MMA,” having the chemical formula CH₂═C(CH₃)CO₂CH₃ and a molecular mass of 100.12 g/mol, is the methyl ester of methacrylic acid (MAA). MMA is used as the monomer for the production of the transparent plastic polymethyl methacrylate (PMMA).

As used herein, the term “(3R)-hydroxybutyl (3R)-hydroxybutyrate” refers to a compound of formula (I):

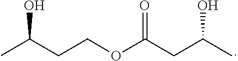

The term (3R)-hydroxybutyl (3R)-hydroxybutyrate is used interchangeably throughout with the terms (R)—(R)-3-hydroxybutyl 3-hydroxybutanoate, (3R)-hydroxybutyl(3R)-hydroxybutyrate, and (R)-3-hydroxybutyl (R)-3-hydroxybutanoate.

As used herein, the term “gene disruption,” or grammatical equivalents thereof, is intended to mean a genetic alteration that renders the encoded gene product inactive. The genetic alteration can be, for example, deletion of the entire gene, deletion of a regulatory sequence required for transcription or translation, deletion of a portion of the gene which results in a truncated gene product, or by any of various mutation strategies that inactivate the encoded gene product. One particularly useful method of gene disruption is complete gene deletion because it reduces or eliminates the occurrence of genetic reversions in the non-naturally occurring microorganisms of the invention.

The non-naturally occurring microbial organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Exemplary metabolic modifications are disclosed herein.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway, reaction, or series of reactions. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having decreased by-products, such as pyruvate by-products (e.g., alanine, and/or valine), TCA derived by-products, acetate and/or ethanol, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. Similarly for a gene disruption, evolutionally related genes can also be disrupted or deleted in a host microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In certain embodiments, provided herein is a non-naturally occurring microbial organism having reduced by-products, that includes a microbial organism having a glycolysis pathway and an enhanced carbon flux through acetyl-CoA. In some embodiments, the microbial organism includes one or more of: (a) an attenuated acetolactate synthase; (b) an acetaldehyde recycling loop; (c) an alanine-recycling loop; and (d) a citrate synthase variant. In some embodiments, the microbial organism includes an attenuated acetolactate synthase. In some embodiments, the microbial organism includes an acetaldehyde recycling loop. In some embodiments, the microbial organism includes an alanine recycling loop. In some embodiments, the microbial organism includes a citrate synthase variant. In some embodiments, the microbial organism includes any citrate synthase variant that reduces TCA activity under microaerobic conditions, e.g., by elevating inhibition by NADH, increasing Km for acetyl-CoA, expression attenuation (e.g., by deletion, knock-down, or reduced expression of citrate synthase (gltA)) or any combination thereof.

In some embodiments, the microbial organism includes two or more of: (a) an attenuated acetolactate synthase; (b) an acetaldehyde recycling loop; (c) an alanine-recycling loop; and (d) a citrate synthase variant. In some embodiments, the microbial organism includes an attenuated acetolactate synthase and an acetaldehyde recycling loop. In some embodiments, the microbial organism includes an attenuated acetolactate synthase and an alanine-recycling loop. In some embodiments, the microbial organism includes an attenuated acetolactate synthase and a citrate synthase variant. In some embodiments, the microbial organism includes an acetaldehyde recycling loop and an alanine recycling loop. In some embodiments, the microbial organism includes an acetaldehyde recycling loop and a citrate synthase variant. In some embodiments, the microbial organism includes an alanine-recycling loop and a citrate synthase variant.

In some embodiments, the microbial organism includes three or more of: (a) an attenuated acetolactate synthase; (b) an acetaldehyde recycling loop; (c) an alanine-recycling loop; and (d) a citrate synthase variant. In some embodiments, the microbial organism includes an attenuated acetolactate synthase, an acetaldehyde recycling loop, and an alanine-recycling loop. In some embodiments, the microbial organism includes an acetaldehyde recycling loop, an alanine recycling loop, and a citrate synthase variant. In some embodiments, the microbial organism includes an attenuated acetolactate synthase, an alanine-recycling loop, and a citrate synthase variant. In some embodiments, the microbial organism includes an attenuated acetolactate synthase, an acetaldehyde recycling loop, and a citrate synthase variant.

In some embodiments, the microbial organism includes each of: (a) an attenuated acetolactate synthase; (b) an acetaldehyde recycling loop; (c) an alanine-recycling loop; and (d) a citrate synthase variant.

Acetolactate synthase (ALS) (Genbank accession numbers ACA79829.1 and ACA79830.1) (EC: 2.2.1.6), also known as acetohydroxyacid synthase (AHAS), catalyzes the first reaction in the pathway for synthesis of branched-chain amino acids. The acetolactate synthase enzyme is at a critical branch point because its reactions determine the extent of carbon flow through to the branched-chain amino acids. The reactions involve the irreversible decarboxylation of pyruvate and the condensation of the acetaldehyde moiety with a second molecule of pyruvate to give 2-acetolactate, or with a molecule of 2-ketobutyrate to yield 2-aceto-2-hydroxybutyrate. Each of the products is then converted further in three reactions, catalyzed by ketol-acid reductoisomerase, dihydroxyacid dehydratase and a transaminase to give valine and isoleucine, respectively. For leucine biosynthesis, four additional enzymes are required using the valine precursor 2-ketoisovalerate as the starting point for synthesis. Accordingly, as provided herein, carbon flow can be directed away from the production of branched-chain amino acids by attenuating acetolactate synthase activity, thereby increasing the carbon flow from pyruvate to acetyl-CoA.

In some organisms, such as E. coli and Salmonella typhimurium, three different acetolactate synthase isozymes may be expressed: AHAS I (encoded by the ilvBN genes), AHAS II (encoded by the ilvGM genes), and AHAS III (encoded by the ilvIH genes). However, because of different chromosomal genetic mutations, AHAS II from E. coli and AHAS III from S. typhimurium can produce inactive proteins. Yet, in some strains of E. Coli, such as Crooks (ATCC 8739), W (ATCC 9637), and B (REL606), ilvG is intact and functional. Therefore, in some embodiments, attenuation of acetolactate synthase involves attenuation of one, two, three, or as many different isozymes that are expressed. In some embodiments, attenuation involves attenuation of the active forms of the acetolactate synthase. In other embodiments, attenuation of acetolactate synthase involves attenuation of all forms of the enzyme.

In some embodiments, the microbial organism includes an attenuated acetolactate synthase where the attenuated acetolactate synthase involves reduced expression of acetolactate synthase. In some embodiments, the amount of reduced expression of acetolactate synthase involves at least about 10% to about 90%. In some embodiments, the amount of reduced expression of acetolactate synthase involves at least about 20% to about 80%. In some embodiments, the amount of reduced expression of acetolactate synthase involves at least about 30% to about 70%. In some embodiments, the amount of reduced expression of acetolactate synthase involves at least about 40% to about 60%. In some embodiments, the amount of reduced expression of acetolactate synthase is about a 50% reduction. In some embodiments, the amount of reduced expression of acetolactate synthase is about a 60% reduction. In some embodiments, the amount of reduced expression of acetolactate synthase is about a 70% reduction. In some embodiments, the amount of reduced expression of acetolactate synthase is about a 80% reduction. In some embodiments, the amount of reduced expression of acetolactate synthase is about a 90% reduction. In some embodiments, the amount of reduced expression of acetolactate synthase is about a 95% reduction. In some embodiments, the amount of reduced expression of acetolactate synthase is about a 100% reduction.

In certain embodiments, the attenuated acetolactate synthase includes a deletion of acetolactate synthase. As described above, in organisms that express multiple forms of the enzyme, the attenuation can include attenuation of one, two, three, or as many different isozymes that are expressed. Therefore, in some embodiments, attenuation of acetolactate synthase includes deletion of one, two, three, or as many different isozymes that are expressed. In some embodiments, attenuation of acetolactate synthase includes deletion of the active forms of the acetolactate synthase. In other embodiments, attenuation of acetolactate synthase involves deletion of all forms of the enzyme.

In some embodiments, the attenuated acetolactate synthase comprises a non-functional acetolactate synthase, such as for example, expression of a dominant negative form of the enzyme. As described above, AHAS II (encoded by the ilvGM genes) from E. coli and AHAS III (encoded by the ilvIH genes) from S. typhimurium can include inactive forms of the proteins. Therefore, in some embodiments, the attenuated acetolactate synthase involves expression of a polynucleotide or a polypeptide encoding an inactive form of acetolactate synthase.

In some embodiments, the attenuated acetolactate synthase includes ilvGM. In some embodiments, the attenuated ilvGM involves a reduced expression of the active form of ilvGM. In specific embodiments, the attenuated ilvGM involves ilvGM deletion of the active form of ilvGM. In some embodiments, microorganism with an attenuated ilvGM is a strain of E. coli with an intact ilvG.

In some embodiments, the attenuated acetolactate synthase decreases the biosynthesis of branched-chain amino acids. In some embodiments, the attenuated acetolactate synthase decreases the biosynthesis of valine, isoleucine, and/or leucine. In some embodiments, the attenuated the attenuated acetolactate synthase decreases the biosynthesis of valine. In some embodiments, the attenuated the attenuated acetolactate synthase decreases the biosynthesis of isoleucine. In some embodiments, the attenuated the attenuated acetolactate synthase decreases the biosynthesis of leucine.

In some embodiments, the non-naturally occurring microbial organism having an attenuated acetolactate synthase reduces the carbon flux into valine by 40 fold. In some embodiments, the non-naturally occurring microbial organism having an attenuated acetolactate synthase reduces the carbon flux into valine by 30 fold. In some embodiments, the non-naturally occurring microbial organism having an attenuated acetolactate synthase reduces the carbon flux into valine by 20 fold. In some embodiments, the non-naturally occurring microbial organism having an attenuated acetolactate synthase reduces the carbon flux into valine by 15 fold. In some embodiments, the non-naturally occurring microbial organism having an attenuated acetolactate synthase reduces the carbon flux into valine by 10 fold. In some embodiments, the non-naturally occurring microbial organism having an attenuated acetolactate synthase reduces the carbon flux into valine by 5 fold. In some embodiments, the non-naturally occurring microbial organism having an attenuated acetolactate synthase reduces the carbon flux into valine by 4 fold. In some embodiments, the non-naturally occurring microbial organism having an attenuated acetolactate synthase reduces the carbon flux into valine by 3 fold. In some embodiments, the non-naturally occurring microbial organism having an attenuated acetolactate synthase reduces the carbon flux into valine by 2 fold. In some embodiments, the non-naturally occurring microbial organism having an attenuated acetolactate synthase reduces the carbon flux into valine by 1.5 fold.

Pyruvate can also undergo transamination to form alanine. Through a two-step process, alanine can be recycled back to form pyruvate. In the first step, L-alanine is converted to D-alanine by alanine racemase (EC 5.1.1.1). The alanine racemase can be constitutively active, or inducible. In E. coli, for example, dadX is responsible for most of the alanine racemase activity in the cell and is inducible by either D-alanine or L-alanine (see, e.g., Wild J, et al., Mol Gen Genet. 1985; 198(2):315-322). Alr, in contrast, is constitutively expressed, but shows a typical dependence upon incubation temperature. In the second step, D-alanine is converted to pyruvate by D-amino acid dehydrogenase. In E. coli and Salmonella typhimurium, for example, dadA generates pyruvate from D-alanine (see, e.g., Wild and Klopotowski, Mol Gen Genet. 1981; 181(3):373-378).

Accordingly, in some embodiments, the present disclosure also provides for the non-naturally occurring microbial organism having an alanine recycling loop that includes at least one exogenous nucleic acid encoding an alanine recycling loop enzyme selected from the group consisting of a D-amino acid dehydrogenase and an alanine racemase. In some embodiments, the alanine recycling loop comprises at least one exogenous nucleic acid encoding a D-amino acid dehydrogenase. In some embodiments, the alanine recycling loop comprises at least one exogenous nucleic acid encoding an alanine racemase. In some embodiments, the alanine recycling loop comprises at least one exogenous nucleic acid encoding a D-amino acid dehydrogenase and an alanine racemase. In specific embodiments, the D-amino acid dehydrogenase is encoded by dadA. In some embodiments, the D-amino acid dehydrogenase is from Escherichia coli str. K-12 substr. MG1655 (NP_415707.1). In certain embodiments, the alanine racemase is encoded by dadX. In certain embodiments, the alanine racemase is from bacteria (NP_747370.1; WP_010955779.1).

As provided herein, a non-naturally occurring microbial organism with an alanine recycling loop is able to redirect carbons that were converted from pyruvate into alanine back to pyruvate. Accordingly, in some embodiments the non-naturally occurring microbial organism having an alanine recycling loop has a reduced alanine concentration, as compared to a microbial organism without an alanine recycling loop. In some embodiments, the reduced alanine concentration is a reduced L-alanine concentration. In some embodiments, the non-naturally occurring microbial organism has reduced a ratio of L-alanine to D-alanine, as compared to a microbial organism without an alanine recycling loop.

In some embodiments, the reduction in alanine concentration is about a 5% to 75% reduction in alanine concentration, as compared to a microbial organism without an alanine recycling loop. In some embodiments, the reduction in alanine concentration is about a 10% to 60% reduction in alanine concentration, as compared to a microbial organism without an alanine recycling loop. In some embodiments, the reduction in alanine concentration is about a 20% to 50% reduction in alanine concentration, as compared to a microbial organism without an alanine recycling loop. In some embodiments, the reduction in alanine concentration is about a 30% to 45% reduction in alanine concentration, as compared to a microbial organism without an alanine recycling loop.

In some embodiments, the reduction in alanine concentration is about a 5% reduction in alanine concentration, as compared to a microbial organism without an alanine recycling loop. In some embodiments, the reduction in alanine concentration is about a 10% reduction in alanine concentration, as compared to a microbial organism without an alanine recycling loop. In some embodiments, the reduction in alanine concentration is about a 15% reduction in alanine concentration, as compared to a microbial organism without an alanine recycling loop. In some embodiments, the reduction in alanine concentration is about a 20% reduction in alanine concentration, as compared to a microbial organism without an alanine recycling loop. In some embodiments, the reduction in alanine concentration is about a 25% reduction in alanine concentration, as compared to a microbial organism without an alanine recycling loop. In some embodiments, the reduction in alanine concentration is about a 30% reduction in alanine concentration, as compared to a microbial organism without an alanine recycling loop. In some embodiments, the reduction in alanine concentration is about a 35% reduction in alanine concentration, as compared to a microbial organism without an alanine recycling loop. In some embodiments, the reduction in alanine concentration is about a 40% reduction in alanine concentration, as compared to a microbial organism without an alanine recycling loop. In some embodiments, the reduction in alanine concentration is about a 45% reduction in alanine concentration, as compared to a microbial organism without an alanine recycling loop. In some embodiments, the reduction in alanine concentration is about a 50% reduction in alanine concentration, as compared to a microbial organism without an alanine recycling loop. In some embodiments, the reduction in alanine concentration is about a 55% reduction in alanine concentration, as compared to a microbial organism without an alanine recycling loop. In some embodiments, the reduction in alanine concentration is about a 60% reduction in alanine concentration, as compared to a microbial organism without an alanine recycling loop. In some embodiments, the reduction in alanine concentration is greater than about 60% reduction in alanine concentration, as compared to a microbial organism without an alanine recycling loop.

As provided herein, decreasing the pyruvate by-products, such by decreasing carbon flux towards the production of alanine and/or branched chain amino acids, can increase the carbon flux through acetyl-CoA and therefore increase the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. Accordingly, in some embodiments, the microorganism having an attenuated acetolactate can have reduced branched chain amino acids, and increased production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the microorganism having an alanine recycling loop can have reduced alanine, and increased production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In some embodiments, the reduction in the production of alanine and/or one or more branched chain amino acids results in about 1 to about 2.5 fold increase in the yield of the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the reduction in the production of alanine and/or one or more branched chain amino acids results in greater than a 2.5 fold increase in the yield of the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In addition, microorganisms that can produce an acetyl-CoA derived product can also have unwanted by-products, such as acetate and/or ethanol that are generated directly from acetyl-CoA. The generation of such by-products can limit the efficiency and/or amount of carbon flow that can be converted to the desired acetyl-CoA derived product, such as 1,3-BDO, MMA, (3R)-hydroxybutyl (3R)-hydroxybutyrate, or any other acetyl-CoA derived product. However, as provided herein, the flow of carbons can be redirected away from the unwanted by-products and towards the desired acetyl-CoA derived product, the present disclosure provides can be enhanced by recycling the unwanted by-products back into acetyl-CoA. In some embodiments, the conversion of unwanted by-products, such as acetate and/or ethanol, back into acetyl-CoA can be achieved by an acetaldehyde recycling loop.

Carbon flow from acetyl-CoA can be converted to unwanted by-products, such as ethanol and/or acetate, by at least two exemplary reactions. In one exemplary reaction, acetyl-CoA is converted to acetaldehyde by CoA-dependent aldehyde dehydrogenase (ALDH; encoded by the ald gene), and acetaldehyde is then converted to ethanol by alcohol dehydrogenase (ADH). Alternatively, in another exemplary reaction acetyl-CoA can be converted to acetate by a CoA hydrolase/thioesterase enzyme.

As provided herein, the present disclosure provides an acetaldehyde recycling loop that can include at least one exogenous nucleic acid encoding an acetaldehyde recycling loop enzyme. In some embodiments, the acetaldehyde recycling loop enzyme is selected from the group consisting of an aldehyde dehydrogenase and an acetyl-CoA synthase.

Aldehyde dehydrogenases (ALDH) (accession WP 000183980.1) (EC: 1.2.1.3) are members of a diverse group of related enzymes catalyzing the oxidation of aldehydes to their corresponding carboxylic acids. In E. coli, for example, more than ten aldehyde dehydrogenase genes have been identified, with some aldehyde dehydrogenase having a preference for certain aldehyde substrates. For example, as provided herein, AldB can preferentially convert acetaldehyde to acetate, and have little to no activity for converting 3HB-aldehyde to 3-hydroxybutyrate.

In some embodiments, the aldehyde dehydrogenase is AldB. In some embodiments, endogenous AldB is expressed at very low levels in the microbial organism. For example, endogenous AldB can be indistinguishable from background levels after detection using LC/MS or using isobaric tags for relative and absolute quantitation (iTRAQ) global proteomics. In some embodiments, the one exogenous nucleic acid encoding an acetaldehyde recycling loop enzyme includes AldB. In certain embodiments, the exogenous nucleic acid is a heterologous nucleic acid.

As provided herein, the acetate produced by the aldehyde dehydrogenase can be further converted to acetyl-CoA by an acetyl-CoA synthase. Acetyl-CoA synthase (accession WP_000078239.1) (EC: 6.2.1.1) catalyzes the ligation of acetate with CoA to produce acetyl-CoA. Thus, in some embodiments, the acetaldehyde recycling loop includes at least one exogenous nucleic acid encoding an acetyl-CoA synthase. In certain embodiments, the acetyl-CoA synthase is an acetyl-CoA synthase variant. In some embodiments, the acetyl-CoA synthase variant can be an acetyl-CoA synthase enzyme that is less sensitive to acetylation, relative to the wild-type acetyl-CoA synthase, but retains acetyl-CoA synthetase activity. One exemplary acetyl-CoA synthase variant is a mutant acetyl-CoA synthase with a replacement of a leucyl residue with a prolyl residue at position 641 (e.g., L641P). In certain embodiments, the acetyl-CoA synthase variant is a Salmonella enterica acetyl CoA synthetase variant. In some embodiments, the acetaldehyde recycling loop includes one or more exogenous nucleic acids encoding both an acetyl-CoA synthase and an aldehyde dehydrogenase. In certain embodiments, the at least one exogenous nucleic acid is a heterologous nucleic acid.

The present disclosure therefore provides an acetaldehyde recycling loop that can reduce the production is unwanted by-products, such as acetate and/or ethanol. In some embodiments, the non-naturally occurring microbial organism has reduced acetate, ethanol, or a combination thereof. In some embodiments, the non-naturally occurring microbial organism has reduced acetate. In some embodiments, the non-naturally occurring microbial organism has reduced ethanol. In some embodiments, the non-naturally occurring microbial organism has reduced acetate, and ethanol.

By decreasing unwanted acetyl-CoA derived by-products, such as acetate and/or ethanol, using for example, an acetaldehyde recycling loop, it is possible to increase the carbon flux from acetyl-CoA through a desired acetyl-CoA derived product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. Accordingly, in some embodiments, the microorganism having an acetaldehyde recycling loop can have reduced acetate and/or ethanol, and increased production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In some embodiments, the reduction in the production of ethanol and/or acetate results in about 1 to about 2.5 fold increase in the yield of the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the reduction in the production of ethanol and/or acetate results in greater than a 2.5 fold increase in the yield of the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

Acetyl-CoA can also be converted to unwanted tricarboxylic acid (TCA) cycle (also known as the citric acid cycle, or the Krebs cycle) derived by-products. The TCA cycle begins with the reaction that combines the acetyl-CoA with oxaloacetic acid to produce citrate. Therefore, the TCA cycle can be responsible for generating unwanted by-products when the desired product is an acetyl-CoA derived product that doesn't involve the TCA cycle, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA,

Citrate synthase is responsible for the rate of reaction in the first step of the cycle when the acetyl-CoA is combined with oxaloacetic acid to form citrate. Citrate synthase is subject to inhibition by NADH, and at least one citrate synthase variant has increased sensitivity to NADH. Wild-type citrate synthase has an arginine (R) at amino acid position 109, whereas the variant contains leucine (L) at amino acid 109 in place of the arginine (R109L) (see Stokell, et al. J Biol Chem. 2003; 278(37):35435-35443). Due to the increased sensitivity to NADH, the citrate synthase variant is less active than the wild-type citrate synthase, and consequently, does not direct as much acetyl-CoA into the TCA cycle.

Accordingly, in some embodiments, the non-naturally occurring microbial organism includes a citrate synthase variant. In some embodiments, the citrate synthase variant is a Type II citrate synthase. In some embodiments, the citrate synthase variant binds NADH with greater affinity than wild-type citrate synthase. In specific embodiments, the citrate synthase variant is encoded by gltA R109L.

As provided herein, the non-naturally occurring microbial organism with a citrate synthase variant has reduced tricarboxylic acid cycle (TCA) cycle derived by-products, as compared to a microbial organism with a wild-type citrate synthase. In some embodiments, the reduction in TCA derived by-products is about a 5% to 75% reduction, as compared to a microbial organism without a citrate synthase variant. In some embodiments, the reduction in TCA derived by-products is about a 10% to 60% reduction, as compared to a microbial organism without a citrate synthase variant. In some embodiments, the reduction in TCA derived by-products is about a 20% to 50% reduction, as compared to a microbial organism without a citrate synthase variant. In some embodiments, the reduction in TCA derived by-products is about a 30% to 45% reduction, as compared to a microbial organism without a citrate synthase variant.

In some embodiments, the reduction in TCA derived by-products is about a 5% reduction, as compared to a microbial organism without a citrate synthase variant. In some embodiments, the reduction in TCA derived by-products is about a 10% reduction, as compared to a microbial organism without a citrate synthase variant. In some embodiments, the reduction in TCA derived by-products is about a 15% reduction, as compared to a microbial organism without a citrate synthase variant. In some embodiments, the reduction in TCA derived by-products is about a 20% reduction, as compared to a microbial organism without a citrate synthase variant. In some embodiments, the reduction in TCA derived by-products is about a 25% reduction, as compared to a microbial organism without a citrate synthase variant. In some embodiments, the reduction in TCA derived by-products is about a 30% reduction, as compared to a microbial organism without a citrate synthase variant. In some embodiments, the reduction in TCA derived by-products is about a 35% reduction, as compared to a microbial organism without a citrate synthase variant. In some embodiments, the reduction in TCA derived by-products is about a 40% reduction, as compared to a microbial organism without a citrate synthase variant. In some embodiments, the reduction in TCA derived by-products is about a 45% reduction, as compared to a microbial organism without a citrate synthase variant. In some embodiments, the reduction in TCA derived by-products is about a 50% reduction, as compared to a microbial organism without a citrate synthase variant. In some embodiments, the reduction in TCA derived by-products is about a 55% reduction, as compared to a microbial organism without a citrate synthase variant. In some embodiments, the reduction in TCA derived by-products is about a 60% reduction, as compared to a microbial organism without a citrate synthase variant. In some embodiments, the reduction in TCA derived by-products is greater than about 60% reduction, as compared to a microbial organism without a citrate synthase variant.

In some embodiments, the reduction in the production of TCA derived by-products results in about 1 to about 2.5 fold increase in the yield of the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the reduction in the production of TCA derived by-products results in greater than a 2.5 fold increase in the yield of the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In some embodiments, the microbial organism includes two or more of: (a) an attenuated acetolactate synthase; (b) an acetaldehyde recycling loop; (c) an alanine-recycling loop; and (d) a citrate synthase variant. In some embodiments, the microbial organism includes three or more of: (a) an attenuated acetolactate synthase; (b) an acetaldehyde recycling loop; (c) an alanine-recycling loop; and (d) a citrate synthase variant. In some embodiments, the microbial organism includes each of: (a) an attenuated acetolactate synthase; (b) an acetaldehyde recycling loop; (c) an alanine-recycling loop; and (d) a citrate synthase variant. The combination of two or more mechanisms for reducing by-products can be additive and allow for an even greater production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the combination of two or more mechanisms for reducing by-products can be synergistic and allow for an even greater production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

As provided herein, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include an 1,3-butanediol (1,3-BDO) pathway, an (3R)-hydroxybutyl (3R)-hydroxybutyrate pathway, a 3-hydroxybutyryl-coenzyme A (3HB-CoA) pathway, a methyl methacrylate (MMA) pathway, an adipate pathway, a caprolactam pathway, a 6-aminocaproic acid (6-ACA) pathway, a hexametheylenediamine (HMDA) pathway, or a methacrylic acid (MAA) pathway. In some embodiments, non-naturally occurring microbial organism having reduced by-products, such as pyruvate by-products (e.g., alanine, and/or valine), TCA derived by-products, acetate and/or ethanol, for enhancing carbon flux through acetyl-CoA comprises a microbial organism having an 1,3-butanediol (1,3-BDO) pathway, a methyl methacrylate (MMA) pathway, a (3R)-hydroxybutyl (3R)-hydroxybutyrate pathway, an amino acid biosynthesis pathway, a 3HB-CoA pathway, a methyl methacrylate (MMA) pathway, an adipate pathway, a caprolactam pathway, a 6-aminocaproic acid (6-ACA) pathway, a hexametheylenediamine (HMDA) pathway, or a methacrylic acid (MAA) pathway.

In certain embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA further includes a 1,3-butanediol (1,3-BDO) pathway. In some embodiments, the 1,3-BDO pathway comprises an enzyme selected from: 1) an acetoacetyl-CoA reductase (CoA-dependent, aldehyde forming), 2) a 3-oxobutyraldehyde reductase (ketone reducing), 3) a 3-hydroxybutyraldehyde reductase, 4) an acetoacetyl-CoA reductase (CoA-dependent, alcohol forming), 5) a 3-oxobutyraldehyde reductase (aldehyde reducing), 6) a 4-hydroxy, 2-butanone reductase, 7) an acetoacetyl-CoA reductase (ketone reducing), 8) a 3-hydroxybutyryl-CoA reductase (aldehyde forming), and 9) a 3-hydroxybutyryl-CoA reductase (alcohol forming). In some embodiments, the 1,3-BDO pathway comprises a nucleic acid encoding an acetoacetyl-CoA reductase (phaB). In specific embodiments, the acetoacetyl-CoA reductase is a mutant acetoacetyl-CoA reductase. In some embodiments, the mutant acetoacetyl-CoA reductase uses NADH as a substrate. Any number of nucleic acids encoding these enzymes can be further introduced into a host microbial organism including one, two, three, four, five, six, seven, eight, up to all nine of the nucleic acids that encode these enzymes. Where one, two, three, four, five, six, seven, or eight exogenous nucleic acids are introduced, such nucleic acids can be any permutation of the additional nine nucleic acids.

In certain embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA further includes a (3R)-hydroxybutyl (3R)-hydroxybutyrate pathway. In some embodiments, the (3R)-hydroxybutyl (3R)-hydroxybutyrate pathway comprises at least one exogenous nucleic acid encoding an enzyme selected from: 1) a (3R)-hydroxybutyl (3R)-hydroxybutyrate ester forming enzyme, 2) a (3R)-hydroxybutyryl-CoA:(R)-1,3-butanediol alcohol transferase, 3) a (3R)hydroxybutyl 3-oxobutyrate ester forming enzyme, 4) an acetoacetyl-CoA:(R)-1,3-butanediol alcohol transferase, 5) a (3R)-hydroxybutyl 3-oxobutyrate reductase, 6) a (3R)-hydroxybutyryl-ACP:(R)-1,3-butanediol ester synthase, and 7) an acetoacetyl-ACP:(R)-1,3-butanediol ester synthase. In some embodiments, the (3R)-hydroxybutyl (3R)-hydroxybutyrate pathway comprises a nucleic acid encoding an acetoacetyl-CoA reductase (phaB). In specific embodiments, the acetoacetyl-CoA reductase is a mutant acetoacetyl-CoA reductase. In some embodiments, the mutant acetoacetyl-CoA reductase uses NADH as a substrate. Any number of nucleic acids encoding these enzymes can be further introduced into a host microbial organism including one, two, three, four, five, six, seven, up to all eight of the nucleic acids that encode these enzymes. Where one, two, three, four, five, six, or seven exogenous nucleic acids are introduced, such nucleic acids can be any permutation of the additional eight nucleic acids.

In certain embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a MMA pathway. In specific embodiments, the MMA pathway comprises at least one exogenous nucleic acid encoding an enzyme selected from: (a) a 4-hydroxybutyryl-CoA dehydratase, a crotonase, a 2-hydroxyisobutyryl-CoA mutase, a 2-hydroxyisobutyryl-CoA dehydratase, and a methacrylic acid (MAA)-CoA: methanol transferase; or (b) a 4-hydroxybutyryl-CoA dehydratase, a crotonase, a 2-hydroxyisobutyryl-CoA mutase, a 3-hydroxyisobutyryl-CoA: methanol transferase, and a methyl-2-hydroxyisobutyrate dehydratase. In some embodiments, the MMA pathway further comprises at least one exogenous nucleic acid encoding an enzyme selected from (c) a methacrylic acid (MAA)-CoA: methanol transferase, a 4-hydroxybutyryl-CoA mutase, and a 3-hydroxyisobutyryl-CoA dehydratase; or (d) a 4-hydroxybutyryl-CoA mutase, a 3-hydroxyisobutyryl-CoA: methanol transferase, and a methyl-3-hydroxyisobutyrate dehydratase.

In other embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a 3HB-CoA pathway. In specific embodiments, the 3HB-CoA pathway comprises at least one exogenous nucleic acid encoding an enzyme selected from: an acetyl-CoA thiolase, and a 3-hydroxybutyryl-CoA dehydrogenase.

In some embodiments, the microbial organism having an increased availability of NADPH can further include a MMA pathway. Production of MMA using microorganisms is known in the art, as exemplified in U.S. Pat. Nos. 9,133,487, and 9,346,902, each of which are incorporated herein by reference in their entirety. In certain embodiments, the MMA pathway comprises an MAA pathway that is then esterified with methanol to produce MMA. In specific embodiments, the MMA pathway comprises at least one exogenous nucleic acid encoding an enzyme selected from (a) a 4-hydroxybutyryl-CoA dehydratase, a crotonase, a 2-hydroxyisobutyryl-CoA mutase, a 2-hydroxyisobutyryl-CoA dehydratase, and a methacrylic acid (MAA)-CoA: methanol transferase; or (b) a 4-hydroxybutyryl-CoA dehydratase, a crotonase, a 2-hydroxyisobutyryl-CoA mutase, a 3-hydroxyisobutyryl-CoA: methanol transferase, and a methyl-2-hydroxyisobutyrate dehydratase. In certain embodiments, the MMA pathway further comprises at least one exogenous nucleic acid encoding an enzyme selected from a second MMA pathway comprising: (c) a methacrylic acid (MAA)-CoA: methanol transferase, a 4-hydroxybutyryl-CoA mutase, and a 3-hydroxyisobutyryl-CoA dehydratase; or (d) a 4-hydroxybutyryl-CoA mutase, a 3-hydroxyisobutyryl-CoA: methanol transferase, and a methyl-3-hydroxyisobutyrate dehydratase.

In certain embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a 6-ACA pathway. In specific embodiments, the 6-ACA pathway comprises at least one exogenous nucleic acid encoding an enzyme selected from 2-amino-7-oxosubarate keto-acid decarboxylase, 2-amino-7-oxoheptanoate decarboxylase, 2-amino-7-oxoheptanoate oxidoreductase, 2-aminopimelate decarboxylase, 6-aminohexanal oxidoreductase, 2-amino-7-oxoheptanoate decarboxylase, or 2-amino-7-oxosubarate amino acid decarboxylase. In other embodiments, the 6-ACA pathway comprises at least one exogenous nucleic acid encoding an enzyme selected from 3-oxo-6-aminohexanoyl-CoA thiolase; 3-oxo-6-aminohexanoyl-CoA reductase; 3-hydroxy-6-aminohexanoyl-CoA dehydratase; 6-aminohex-2-enoyl-CoA reductase; and 6-aminocaproyl-CoA/acyl-CoA transferase, 6-aminocaproyl-CoA synthase, or 6-aminocaproyl-CoA hydrolase.

In other embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a caprolactam pathway. In specific embodiments, the caprolactam pathway comprises at least one exogenous nucleic acid encoding an enzyme selected from 3-oxoadipyl-CoA thiolase, 3-oxoadipyl-CoA reductase, 3-hydroxyadipyl-CoA dehydratase, 5-carboxy-2-pentenoyl-CoA reductase, adipyl-CoA reductase (aldehyde forming), 6-aminocaproate transaminase, 6-aminocaproate dehydrogenase, 6-aminocaproyl-CoA/acyl-CoA transferase, and 6-aminocaproyl-CoA synthase.

In certain embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include an adipate pathway. In specific embodiments, the adipate pathway comprises at least one exogenous nucleic acid encoding an enzyme selected from 3-oxoadipyl-CoA thiolase, 3-oxoadipyl-CoA reductase, 3-hydroxyadipyl-CoA dehydratase, 5-carboxy-2-pentenoyl-CoA reductase, adipyl-CoA hydrolase, adipyl-CoA ligase, adipyl-CoA transferase and phosphotransadipylase/adipate kinase.

In other embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a hexamethylenediamine (HMDA) pathway. In specific embodiments, the HMDA pathway comprises at least one exogenous nucleic acid encoding an enzyme selected from 3-oxoadipyl-CoA thiolase, 3-oxoadipyl-CoA reductase, 3-hydroxyadipyl-CoA dehydratase, 5-carboxy-2-pentenoyl-CoA reductase, adipyl-CoA reductase (aldehyde forming), 6-aminocaproate transaminase, 6-aminocaproate dehydrogenase, 6-aminocaproyl-CoA/acyl-CoA transferase, 6-aminocaproyl-CoA synthase, 6-aminocaproyl-CoA reductase (aldehyde forming), HMDA transaminase, and HMDA dehydrogenase. In other embodiments, the HMDA pathway comprises at least one exogenous nucleic acid encoding an enzyme selected from 6-aminocaproyl-CoA/acyl-CoA transferase or 6-aminocaproyl-CoA synthase; 6-aminocaproyl-CoA reductase (aldehyde forming); and hexamethylenediamine transaminase or hexamethylenediamine dehydrogenase.

In some embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a MAA pathway. In specific embodiments, the MAA pathway comprises at least one exogenous nucleic acid encoding an enzyme selected from (1) (i) a succinyl-CoA transferase, ligase, or synthetase; (ii) a methylmalonyl-CoA mutase; (iii) a methylmalonyl-CoA epimerase; (iv) a methylmalonyl-CoA reductase (aldehyde forming); (v) a methylmalonate semialdehyde reductase; and (vi) a 3-hydroxyisobutyrate dehydratase; (2) (i) a succinyl-CoA transferase, ligase, or synthetase; (ii) a methylmalonyl-CoA mutase; (iii) a methylmalonyl-CoA reductase (aldehyde forming); (iv) a methylmalonate semialdehyde reductase; and (v) a 3-hydroxyisobutyrate dehydratase; or (3) (i) a succinyl-CoA transferase, ligase, or synthetase; (ii) a methylmalonyl-CoA mutase; (iii) a methylmalonyl-CoA reductase (alcohol forming); and (iv) a 3-hydroxyisobutyrate dehydratase.

In some embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a 1,3-butanediol pathway. In some embodiments, the 1,3-butanediol pathway includes at least one exogenous nucleic acid encoding an enzyme or protein that converts a substrate to a product selected from the group consisting of glucose to pyruvate, pyruvate to acetyl-CoA, acetyl-CoA to acetoacetyl-CoA, acetoacetyl-CoA to 3-hydroxybutyryl-CoA, 3-hydroxybutyryl-CoA to 3-hydroxybutryaldehyde, and 3-hydroxybutryaldehyde to 1,3-BDO.

In some embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a (3R)-hydroxybutyl (3R)-hydroxybutyrate pathway. In some embodiments, the (3R)-hydroxybutyl (3R)-hydroxybutyrate pathway includes at least one exogenous nucleic acid encoding an enzyme or protein that converts a substrate to a product selected from the group consisting of (R)-1,3-butanediol and (3R)-hydroxybutyrate to (3R)-hydroxybutyl (3R)-hydroxybutyrate, (R)-1,3-butanediol and (3R)-hydroxybutyryl-CoA to (3R)-hydroxybutyl (3R)-hydroxybutyrate, (R)-1,3-butanediol and (3R)-hydroxybutyl-ACP to (3R)-hydroxybutyl (3R)-hydroxybutyrate, (R)-1,3-butanediol and acetoacetate to (3R)-hydroxybutyl 3-oxobutyrate, (R)-1,3-butanediol and acetoacetyl-CoA to (3R)-hydroxybutyl 3-oxobutyrate, (R)-1,3-butanediol and acetoacetyl-ACP to (3R)-hydroxybutyl 3-oxobutyrate, (3R)-hydroxybutyl 3-oxobutyrate to (3R)-hydroxybutyl (3R)-hydroxybutyrate, acetyl-CoA to malonyl-CoA, malonyl-CoA to acetoacetyl-CoA, acetyl-CoA to acetoacetyl-CoA, acetoacetyl-CoA to acetoacetate, acetoacetyl-CoA to (3 S)-hydroxybutyryl-CoA, (3 S)-hydroxybutyryl-CoA to (3R)-hydroxybutyryl-CoA, (3R)-hydroxybutyryl-CoA to (3R)-hydroxybutyrate, (3R)-hydroxybutyryl-CoA to (3R)-hydroxybutyraldehyde, (3R)-hydroxybutyrate to (3R)-hydroxybutyraldehyde, (3R)-hydroxybutyraldehyde to (R)-1,3-butanediol, malonyl-ACP to acetoacetyl-ACP, acetoacetyl-ACP to acetoacetyl-CoA, acetoacetyl-ACP to (3R)-hydroxybutyryl-ACP, (3R)-hydroxybutyryl-ACP to (3R)-hydroxybutyryl-CoA, (3R)-hydroxybutyryl-ACP to (3R)-hydroxybutyrate, (3R)-hydroxybutyryl-ACP to (3R)-hydroxybutyraldehyde, and (3R)-hydroxybutyryl-ACP to (R)-1,3-butanediol. One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of a (3R)-hydroxybutyl (3R)-hydroxybutyrate pathway, such as those disclosed in U.S. application Ser. No. 14/893,510, published as U.S. 2016-0108442 A1, which is incorporated herein by reference in its entirety.

In some embodiments, the non-naturally occurring microbial organism having reduced by-products, such as pyruvate by-products (e.g., alanine, and/or valine), TCA derived by-products, acetate and/or ethanol, for enhancing carbon flux through acetyl-CoA can further include a MMA pathway. In some embodiments, the MMA pathway includes at least one exogenous nucleic acid encoding an enzyme or protein that converts a substrate to a product selected from the group consisting of 4-HB-CoA to crotonyl-CoA and 3HB-CoA, crotonyl-CoA to 3HB-CoA, 3HB-CoA to MAA-CoA or methyl-3HB, and MAA-CoA or methyl-3HB to MMA. In a further embodiment, the invention provides a non-naturally occurring microbial organism having a MMA pathway, wherein the non-naturally occurring microbial organism comprises at least one exogenous nucleic acid encoding an enzyme or protein that converts a substrate to a product selected from the group consisting of 4HB-CoA to crotonyl-CoA, crotonyl-CoA to (3R)—HB-CoA or (3 S)—HB-CoA, (3R)—HB-CoA or (3 S)—HB-CoA to 2-HIB-CoA, 2-HIB-CoA to MAA-CoA or 2HB-Me, and MAA-CoA or 2HB-Me to MMA. In yet a further embodiment, the invention provides a non-naturally occurring microbial organism having a 1,3-BDO pathway, wherein the non-naturally occurring microbial organism comprises at least one exogenous nucleic acid encoding an enzyme or protein that converts a substrate to a product selected from the group consisting of 4HB-CoA to crotonyl-CoA, crotonyl-CoA to (3R)—HB-CoA or (3S)-HB-CoA, (3R)—HB-CoA or (3S)—HB-CoA to (3R)- or (3S)-1,3 BDO.

In an additional embodiment, the invention provides a non-naturally occurring microbial organism having a methacrylic acid pathway, wherein the non-naturally occurring microbial organism comprises at least one exogenous nucleic acid encoding an enzyme or protein that converts a substrate to a product selected from the group consisting of acetyl-CoA and pyruvate to citramalate, citramalate to citraconate, and citraconate to methacrylate; acetyl-CoA and pyruvate to citramalyl-CoA, citramalyl-CoA to citramalate, citramalate to citraconate, and citraconate to methyacrylate; aconitate to itaconate, itaconate to itaconyl-CoA, itaconyl-CoA to citramalyl-CoA, citramalyl-CoA to citramalate, citramalate to mesaconate, mesaconate to methacrylate, and so forth such as the reactions described herein. One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of a methacrylic acid pathway, such as the pathway described herein. Additionally provided is a methacrylic acid pathway comprising acetoacetyl-CoA thiolase, acetoacetyl-CoA reductase, crotonase, 4-hydroxybutyryl-CoA dehydratase (or crotonyl-CoA hydratase, 4-hydroxy), 4-hydroxybutyryl-CoA mutase, 3-hydroxyisobutyryl-CoA synthetase or 3-hydroxyisobutyryl-CoA hydrolase or 3-hydroxyisobutyryl-CoA transferase, and 3-hydroxyisobutyrate dehydratase. Also provided is a methacrylic acid pathway comprising acetoacetyl-CoA thiolase, acetoacetyl-CoA reductase, crotonase, 4-hydroxybutyryl-CoA dehydratase, 4-hydroxybutyryl-CoA mutase, 3-hydroxyisobutyryl-CoA dehydratase, and methacrylyl-CoA synthetase or methacrylyl-CoA hydrolase or methacrylyl-CoA transferase. The production of MAA is known in the art and can be found, for example, in U.S. application Ser. No. 13/436,811, published as U.S. 2013-0065279A1, which is incorporated herein by reference in its entirety.

In an additional embodiment, the invention provides a non-naturally occurring microbial organism having a caprolactam pathway, wherein the non-naturally occurring microbial organism comprises at least one exogenous nucleic acid encoding an enzyme or protein that converts a substrate to a product selected from the group consisting of adipyl-CoA to adipate, adipyl-CoA to adipate semialdehyde, adipate to adipate semialdehyde, adipate semialdehyde to 6-hydroxyhexanoate, 6-hydroxyhexanoate to 6-hydroxyhexanoyl-CoA, 6-hydroxyhexanoate to 6-hydroxyhexanoyl-phosphate, 6-hydroxyhexanoate to caprolactone, 6-hydroxyhexanoyl-CoA to 6-hydroxyhexanoyl phosphate, 6-hydroxyhexanoyl phosphate to caprolactone, 6-hydroxyhexanoyl-CoA to caprolactone, 4-hydroxybutyryl-CoA to 3-oxo-6-hydroxy hexanoyl-CoA, to 3-oxo-6-hydroxy hexanoyl-CoA to 3,6-dihydroxy hexanoyl-CoA, 3,6-dihydroxy hexanoyl-CoA to 6-hydroxyhex-2-enoyl-CoA, 6-hydroxyhex-2-enoyl-CoA to 6-hydroxyhexanoyl-CoA, 6-hydroxyhexanoyl-CoA to 6-hydroxyhexanoate, cyclohexanon to caprolactone, adipate semialdehyde to cyclohexane-1,2-dione, cyclohexane-1,2-dione to 2-hydroxycyclohexanone, to 2-hydroxycyclohexanone to cyclohexane-1,2-diol, cyclohexane-1,2-diol to cyclohexone, pimeloyl-CoA to 2-ketocyclohexone-1-carboxoyl-CoA, 2-ketocyclohexone-1-carboxoyl-CoA to 2-ketocyclohexane-1-carboxylate, 2-ketocyclohexane-1-carboxylate to cyclohexanone, cyclohexanone to cyclohexanoxime, and cyclohexanoxime to caprolactam. One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of a caprolactam pathway.

In some embodiments, the non-naturally occurring microbial organism provided herein having reduced by-products, such as pyruvate by-products (e.g., alanine, and/or valine), TCA derived by-products, acetate and/or ethanol, for enhancing carbon flux through acetyl-CoA can further include an adipate pathway, wherein the microbial organism contains at least one exogenous nucleic acid encoding a polypeptide that converts a substrate to a product selected from succinyl-CoA and acetyl-CoA to 3-oxoadipyl-CoA; 3-oxoadipyl-CoA to 3-hydroxyadipyl-CoA; 3-hydroxyadipyl-CoA to 5-carboxy-2-pentenoyl-CoA; 5-carboxy-2-pentenoyl-CoA to adipyl-CoA; adipyl-CoA to adipate (see, e.g., WO2012/177721, which is incorporated herein in its entirety). Additionally, a non-naturally occurring microbial organism can have an adipate pathway, wherein the microbial organism contains at least one exogenous nucleic acid encoding a polypeptide that converts a substrate to a product selected from succinyl-CoA and acetyl-CoA to 3-oxoadipyl-CoA; 3-oxoadipyl-CoA to 3-oxoadipate; 3-oxoadipate to 3-hydroxyadipate; 3-hydroxyadipate to hexa-2-enedioate (also referred to herein as 5-carboxy-2-pentenoate); hexa-2-enedioate to adipate. Also, a non-naturally occurring microbial organism can have a 6-aminocaproic acid pathway, wherein the microbial organism contains at least one exogenous nucleic acid encoding a polypeptide that converts a substrate to a product selected from adipyl-CoA to adipate semialdehyde; and adipate semialdehyde to 6-aminocaproate. Furthermore, a non-naturally occurring microbial organism can have a caprolactam pathway, wherein the microbial organism contains at least one exogenous nucleic acid encoding a polypeptide that converts a substrate to a product selected from adipyl-CoA to adipate semialdehyde; adipate semialdehyde to 6-aminocaproate; and 6-aminocaproate to caprolactam. Additionally, a non-naturally occurring microbial organism can have an adipate pathway, wherein the microbial organism contains at least one exogenous nucleic acid encoding a polypeptide that converts a substrate to a product selected from alpha-ketoadipate to alpha-ketoadipyl-CoA; alpha-ketoadipyl-CoA to 2-hydroxyadipyl-CoA; 2-hydroxyadipyl-CoA to 5-carboxy-2-pentenoyl-CoA; 5-carboxy-2-pentenoyl-CoA to adipyl-CoA; and adipyl-CoA to adipate. Also, a non-naturally occurring microbial organism can have an adipate pathway, wherein the microbial organism contains at least one exogenous nucleic acid encoding a polypeptide that converts a substrate to a product selected from alpha-ketoadipate to 2-hydroxyadipate; 2-hydroxyadipate to 2-hydroxyadipyl-CoA; 2-hydroxyadipyl-CoA to 5-carboxy-2-pentenoyl-CoA; 5-carboxy-2-pentenoyl-CoA to adipyl-CoA; and adipyl-CoA to adipate.

Additionally, a non-naturally occurring microbial organism can have a 6-aminocaproyl-CoA pathway, wherein the microbial organism contains at least one exogenous nucleic acid encoding a polypeptide that converts a substrate to a product selected from 4-aminobutyryl-CoA and acetyl-CoA to 3-oxo-6-aminohexanoyl-CoA; 3-oxo-6-aminohexanoyl-CoA to 3-hydroxy-6-aminohexanoyl-CoA; 3-hydroxy-6-aminohexanoyl-CoA to 6-aminohex-2-enoyl-CoA; 6-aminohex-2-enoyl-CoA to 6-aminocaproyl-CoA. Additional substrates and products of such a pathway can include 6-aminocaproyl-CoA to 6-aminocaproate; 6-aminocaproyl-CoA to caprolactam; or 6-aminocaproyl-CoA to 6-aminocaproate semialdehyde and 6-aminocaproate semialdehyde to hexamethylenediamine. A non-naturally occurring microbial organism also can have a 6-aminocaproic acid pathway, wherein the microbial organism contains at least one exogenous nucleic acid encoding a polypeptide that converts a substrate to a product selected from 4-aminobutyryl-CoA and acetyl-CoA to 3-oxo-6-aminohexanoyl-CoA; 3-oxo-6-aminohexanoyl-CoA to 3-oxo-6-aminohexanoate; 3-oxo-6-aminohexanoate to 3-hydroxy-6-aminohexanoate; 3-hydroxy-6-aminohexanoate to 6-aminohex-2-enoate; and 6-aminohex-2-enoate to 6-aminocaproate. Additional substrates and products of such a pathway can include 6-aminocaproate to caprolactam or 6-aminocaproate to 6-aminocaproyl-CoA, 6-aminocaproyl-CoA to 6-aminocaproate semialdehyde, and 6-aminocaproate semialdehyde to hexamethylenediamine.

Additionally, a non-naturally occurring microbial organism can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 2-oxohept-4-ene-1,7-dioate (OHED): 2-oxohept-4-ene-1,7-dioate (OHED) to 2-oxoheptane-1,7-dioate (2-OHD); 2-oxoheptane-1,7-dioate (2-OHD) to adipate semialdehyde; and adipate semialdehyde to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 2-oxohept-4-ene-1,7-dioate (OHED); 2-oxohept-4-ene-1,7-dioate (OHED) to 6-oxohex-4-enoate (6-OHE): 6-oxohex-4-enoate (6-OHE) to adipate semialdehyde; and adipate semialdehyde to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 2-oxohept-4-ene-1,7-dioate (OHED); 2-oxohept-4-ene-1,7-dioate (OHED) to 2-aminohept-4-ene-1,7-dioate (2-AHE); 2-aminohept-4-ene-1,7-dioate (2-AHE) to 2-aminoheptane-1,7-dioate (2-AHD); and 2-aminoheptane-1,7-dioate (2-AHD) to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 2-oxohept-4-ene-1,7-dioate (OHED); 2-oxohept-4-ene-1,7-dioate (OHED) to 2-oxoheptane-1,7-dioate (2-OHD); 2-oxoheptane-1,7-dioate (2-OHD) to 2-aminoheptane-1,7-dioate (2-AHD); and 2-aminoheptane-1,7-dioate (2-AHD) to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 3-hydroxyadipyl-CoA; 3-hydroxyadipyl-CoA to 2,3-dehydroadipyl-CoA; 2,3-dehydroadipyl-CoA to adipyl-CoA; adipyl-CoA to adipate semialdehyde; and adipate semialdehyde to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 2-oxohept-4-ene-1,7-dioate (OHED); 2-oxohept-4-ene-1,7-dioate (OHED) to 2,3-dehydroadipyl-CoA; 2,3-dehydroadipyl-CoA to adipyl-CoA; adipyl-CoA to adipate semialdehyde; and adipate semialdehyde to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from pyruvate and succinic semialdehyde to 4-hydroxy-2-oxoheptane-1,7-dioate; 4-hydroxy-2-oxoheptane-1,7-dioate (HODH) to 2-oxohept-4-ene-1,7-dioate (OHED); 2-oxohept-4-ene-1,7-dioate (OHED) to 2-oxoheptane-1,7-dioate (2-OHD); 2-oxoheptane-1,7-dioate (2-OHD) to adipyl-CoA; adipyl-CoA to adipate semialdehyde; and adipate semialdehyde to 6-aminocaproate.

Additionally, a non-naturally occurring microbial organism can have a 6-aminocaproic acid pathway, wherein the microbial organism contains at least one exogenous nucleic acid encoding a polypeptide that converts a substrate to a product selected from glutamate to glutamyl-CoA; glutamyl-coA to 3-oxo-6-amino-pimeloyl-CoA; 3-oxo-6-amino-pimeloyl-CoA to 3-hydroxy-6-amino-pimeloyl-CoA; 3-hydroxy-6-amino-pimeloyl-CoA to 6-amino-7-carboxy-hept-2-enoyl-CoA; 6-amino-7-carboxy-hept-2-enoyl-CoA to 6-aminopimeloyl-CoA; 6-aminopimeloyl-CoA to 2-aminopimelate; and 2-aminopimelate to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 2-aminopimelate; and 2-aminopimelate to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from homolysine to 6-aminohexanamide; and 6-aminohexanamide to 6-aminocaproate. A non-naturally occurring microbial organism alternatively can have a 6-aminocaproic acid pathway, wherein the microbial organism contains at least one exogenous nucleic acid encoding a polypeptide that converts a substrate to a product selected from adipate to adipate semialdehyde; adipate to adipylphospate; and adipylphospate to adipate semialdehyde.

Additionally, a non-naturally occurring microbial organism can have a 6-aminocaproic acid pathway, wherein the microbial organism contains at least one exogenous nucleic acid encoding a polypeptide that converts a substrate to a product selected from 2-amino-7-oxosubarate to 2-amino-7-oxoheptanoate; 2-amino-7-oxoheptanoate to 6-aminohexanal; 6-aminohexanal to 6-aminocaproate; 2-amino-7-oxosubarate to 2-amino-7-oxoheptanoate; 2-amino-7-oxoheptanoate to 6-aminohexanal; 2-amino-7-oxoheptanoate to 2-aminopimelate; and 2-aminopimelate to 6-aminocaproate. A non-naturally occurring microbial organism can further have a 2-amino-7-oxosubarate pathway, wherein the microbial organism contains at least one exogenous nucleic acid encoding a polypeptide that converts a substrate to a product selected from glutamate-5-semialdehyde to 2-amino-5-hydroxy-7-oxosubarate; 2-amino-5-hydroxy-7-oxosubarate to 2-amino-5-ene-7-oxosubarate; and 2-amino-5-ene-7-oxosubarate to 2-amino-7-oxosubarate.

Additionally, a non-naturally occurring microbial organism can have an hexamethylenediamine (HMDA) pathway, wherein the microbial organism contains at least one exogenous nucleic acid encoding a polypeptide that converts a substrate to a product selected from 6-aminocaproate to [(6-aminohexanoyl)oxy]phosphonate (6-AHOP); [(6-aminohexanoyl)oxy]phosphonate (6-AHOP) to 6-aminocaproaic semialdehyde; and 6-aminocaproaic semialdehyde to hexamethylenediamine. A non-naturally occurring microbial organism alternatively can have a HMDA pathway, wherein the microbial organism contains at least one exogenous nucleic acid encoding a polypeptide that converts a substrate to a product selected from 6-aminocaproate to [(6-aminohexanoyl)oxy]phosphonate (6-AHOP); [(6-aminohexanoyl)oxy]phosphonate (6-AHOP) to 6-aminocaproyl-CoA; 6-aminocaproyl-CoA to 6-aminocaproaic semialdehyde; and 6-aminocaproaic semialdehyde to hexamethylenediamine. A non-naturally occurring microbial organism alternatively can have a HMDA pathway, wherein the microbial organism contains at least one exogenous nucleic acid encoding a polypeptide that converts a substrate to a product selected from 6-aminocaproate to 6-aminocaproyl-CoA; 6-aminocaproyl-CoA to 6-aminocaproic semialdehyde; and 6-aminocaproic semialdehyde to hexamethylenediamine. A non-naturally occurring microbial organism alternatively can have a HMDA pathway, wherein the microbial organism contains at least one exogenous nucleic acid encoding a polypeptide that converts a substrate to a product selected from 6-aminocaproate to 6-acetamidohexanoate; 6-acetamidohexanoate to [(6-acetamidohexanoy)oxy]phosphonate (6-AAHOP); [(6-acetamidohexanoy)oxy]phosphonate (6-AAHOP) to 6-acetamidohexanal; 6-acetamidohexanal to 6-acetamidohexanamine; and 6-acetamidohexanamine to hexamethylenediamine. A non-naturally occurring microbial organism alternatively can have a HMDA pathway, wherein the microbial organism contains at least one exogenous nucleic acid encoding a polypeptide that converts a substrate to a product selected from 6-aminocaproate to 6-acetamidohexanoate; 6-acetamidohexanoate to 6-acetamidohexanoyl-CoA; 6-acetamidohexanoyl-CoA to 6-acetamidohexanal; 6-acetamidohexanal to 6-acetamidohexanamine; and 6-acetamidohexanamine to hexamethylenediamine. A non-naturally occurring microbial organism alternatively can have a HMDA pathway, wherein the microbial organism contains at least one exogenous nucleic acid encoding a polypeptide that converts a substrate to a product selected from 6-aminocaproate to 6-acetamidohexanoate; 6-acetamidohexanoate to [(6-acetamidohexanoy)oxy]phosphonate (6-AAHOP); [(6-acetamidohexanoy)oxy]phosphonate (6-AAHOP) to 6-acetamidohexanoyl-CoA; 6-acetamidohexanoyl-CoA to 6-acetamidohexanal; 6-acetamidohexanal to 6-acetamidohexanamine; and 6-acetamidohexanamine to hexamethylenediamine.

Additionally, a non-naturally occurring microbial organism can have an hexamethylenediamine (HMDA) pathway, wherein the microbial organism contains at least one exogenous nucleic acid encoding a polypeptide that converts a substrate to a product selected from glutamate to glutamyl-CoA; glutamyl-coA to 3-oxo-6-amino-pimeloyl-CoA; 3-oxo-6-amino-pimeloyl-CoA to 3-hydroxy-6-amino-pimeloyl-CoA; 3-hydroxy-6-amino-pimeloyl-CoA to 6-amino-7-carboxy-hept-2-enoyl-CoA; 6-amino-7-carboxy-hept-2-enoyl-CoA to 6-aminopimeloyl-CoA; 6-aminopimeloyl-CoA to 2-amino-7-oxoheptanoate; -amino-7-oxoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-oxo-1-carboxy heptanal; 3-oxo-1-carboxy heptanal to 3-oxo-7-amino heptanoate; 3-oxo-7-amino heptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 5-oxopimeloyl phosponate; 5-oxopimeloyl phosponate to 3-oxo-1-carboxy heptanal; 3-oxo-1-carboxy heptanal to 3-oxo-7-amino heptanoate; 3-oxo-7-amino heptanoate to 3,7-diamino heptanoate; 3,7-diamino heptanoate to homolysine and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 5-oxopimeloyl-CoA; 5-oxopimeloyl-CoA to 3-oxo-1-carboxy heptanal; 3-oxo-1-carboxy heptanal to 3-oxo-7-amino heptanoate; 3-oxo-7-amino heptanoate to 3,7-diamino heptanoate; 3,7-diamino heptanoate to homolysine and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-oxo-1-carboxy heptanal; 3-oxo-1-carboxy heptanal to 3-amino-7-oxoheptanoate; 3-amino-7-oxoheptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 5-oxopimeloyl-CoA; 5-oxopimeloyl-CoA to 3-oxo-1-carboxy heptanal; 3-oxo-1-carboxy heptanal to 3-amino-7-oxoheptanoate; 3-amino-7-oxoheptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 5-oxopimeloyl phosponate; 5-oxopimeloyl phosponate to 3-oxo-lcarboxy heptanal; 3-oxo-1-carboxy heptanal to 3-amino-7-oxoheptanoate; 3-amino-7-oxoheptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 3-amino-7-oxoheptanoate; 3-amino-7-oxoheptanoate to 2-amino-7-axoheptanoate; 2-amino-7-axoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 5-aminopimeloyl phosphonate; 5-aminopimeloyl phosphonate to 3-amino-7-oxoheptanoate; 3-amino-7-oxoheptanoate to 2-amino-7-axoheptanoate; 2-amino-7-axoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 5-aminopimeloyl-CoA; 5-aminopimeloyl-CoA to 3-amino-7-oxoheptanoate; 3-amino-7-oxoheptanoate to 2-amino-7-axoheptanoate; 2-amino-7-axoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 3-amino-7-oxoheptanoate; 3-amino-7-oxoheptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 5-aminopimeloyl-CoA; 5-aminopimeloyl-CoA to 3-amino-7-oxoheptanoate; 3-amino-7-oxoheptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 5-aminopimeloyl phosphonate; 5-aminopimeloyl phosphonate to 3-amino-7-oxoheptanoate; 3-amino-7-oxoheptanoate to 3,7-diamino heptanoate; 3,7-diamino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 2-aminopimelate; 2-aminopimelate to 2-amino-7-oxoheptanoate; 2-amino-7-oxoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 2-aminopimelate; 2-aminopimelate to 6-aminopimeloylphosphonate; 6-aminopimeloylphosphonate to 2-amino-7-oxoheptanoate; 2-amino-7-oxoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from glutaryl-CoA to 3-oxopimeloyl-CoA; 3-oxopimeloyl-CoA to 3-oxopimelate; 3-oxopimelate to 3-aminopimelate; 3-aminopimelate to 2-aminopimelate; 2-aminopimelate to 6-aminopimeloyl-CoA; 6-aminopimeloyl-CoA to 2-amino-7-oxoheptanoate; 2-amino-7-oxoheptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from pyruvate and 4-aminobutanal to 2-oxo-4-hydroxy 7-aminoheptanoate; 2-oxo-4-hydroxy 7-aminoheptanoate to 2-oxo-7-amino hept-3-enoate; 2-oxo-7-amino hept-3-enoate to 2-oxo-7-amino heptanoate; 2-oxo-7-amino heptanoate to homolysine; and homolysine to HMDA. A non-naturally occurring microbial organism alternatively can have a HMDA pathway, wherein the microbial organism contains at least one exogenous nucleic acid encoding a polypeptide that converts a substrate to a product selected from pyruvate and 4-aminobutanal to 2-oxo-4-hydroxy 7-aminoheptanoate; 2-oxo-4-hydroxy 7-aminoheptanoate to 2-oxo-7-amino hept-3-enoate; 2-oxo-7-amino hept-3-enoate to 2-oxo-7-amino heptanoate; 2-oxo-7-aminoheptanoate to 6-aminohexanal; and 6-aminohexanal to HMDA. A non-naturally occurring microbial organism alternatively can have a HMDA pathway, wherein the microbial organism contains at least one exogenous nucleic acid encoding a polypeptide that converts a substrate to a product selected from 6-aminocaproate to 6-aminocaproic semialdehyde; and 6-aminocaproic semialdehyde to HMDA. A non-naturally occurring microbial organism alternatively can have a HMDA pathway, wherein the microbial organism contains at least one exogenous nucleic acid encoding a polypeptide that converts a substrate to a product selected from 6-aminocaproate to 6-acetamidohexanoate; 6-acetamidohexanoate to 6-acetamidohexanal; 6-acetamidohexanal to 6-acetamidohexanamine; 6-acetamidohexanamine to HMDA. A non-naturally occurring microbial organism alternatively can have a HMDA pathway, wherein the microbial organism contains at least one exogenous nucleic acid encoding a polypeptide that converts a substrate to a product selected from 2-amino-7-oxosubarate to 2-amino-7-oxoheptanoate; 2-amino-7-oxoheptanoate to 6-aminohexanal; 6-aminohexanal to HMDA; 2-amino-7-oxosubarate to 2-oxo-7-aminoheptanoate; 2-amino-7-oxoheptanoate to homolysine; homolysine to HMDA; 2-oxo-7-aminoheptanoate to homolysine; 2-oxo-7-aminoheptanoate to 6-aminohexanal; 2-amino-7-oxosubarate to 2,7-diaminosubarate; and 2,7-diaminosubarate to homolysine. A non-naturally occurring microbial organism can further have a 2-amino-7-oxosubarate pathway, wherein the microbial organism contains at least one exogenous nucleic acid encoding a polypeptide that converts a substrate to a product selected from glutamate-5-semialdehyde to 2-amino-5-hydroxy-7-oxosubarate; 2-amino-5-hydroxy-7-oxosubarate to 2-amino-5-ene-7-oxosubarate; and 2-amino-5-ene-7-oxosubarate to 2-amino-7-oxosubarate.

One skilled in the art will understand that these are merely exemplary and that any of the substrate-product pairs disclosed herein suitable to produce a desired acetyl-CoA derived product and for which an appropriate activity is available for the conversion of the substrate to the product can be readily determined by one skilled in the art based on the teachings herein.

It is also understood that the expression of the exogenous nucleic acids disclosed herein can be regulated by various promoters, such as an endogenous promoter, a constitutive promoter, or an inducible promoter. A person of average skill in the art would understand which type of promoter to use given the level and duration of expression desired. For example, if the level of expression desired was the endogenous level, a person of average skill would understand that an endogenous promoter could be used to control the expression of the exogenous nucleic acid. Further, if the expression was desired to be, for example, temporary or at a specific point during fermentation, an endogenous promoter could be used to control the expression of the exogenous nucleic acid. However, if the expression was desired to be, for example, constant and robust, a constitutive promoter could be used to control the expression of the exogenous nucleic acid. Therefore, in some embodiments, the exogenous nucleic acid encoding an enzyme is regulated by an endogenous promoter, a constitutive promoter, or an inducible promoter.

While generally described herein as a microbial organism having reduced by-products, such as pyruvate by-products (e.g., alanine, and/or valine), TCA derived by-products, acetate and/or ethanol, for enhancing carbon flux through acetyl-CoA, it is understood that the invention additionally provides a non-naturally occurring microbial organism comprising at least one exogenous nucleic acid encoding an enzyme expressed in a sufficient amount to increase availability of the production of a desired acetyl-CoA derived product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, as well as intermediates derived therefrom.

Accordingly, it is understood that the invention additionally provides in some embodiments a non-naturally occurring microbial organism that includes an intermediate of an 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA biosynthesis pathway, wherein the pathway contains at least one enzyme that acts on an acetyl-CoA derived product, and the pathway enzyme is expressed in a sufficient amount to produce an intermediate of a 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA pathway. Therefore, in addition to a microbial organism containing an 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA biosynthesis pathway that produces 1,3-BDO, (3R)-hydroxybutyl (3R)-hydroxybutyrate, MMA, or amino acid, the invention additionally provides a non-naturally occurring microbial organism, where the microbial organism produces an intermediate of an acetyl-CoA derived product, such as an intermediate of an 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA biosynthesis pathway.

It is understood that any of the pathways disclosed herein, as described throughout and incorporated by reference in their entirety, can be utilized by the non-naturally occurring microbial organism having reduced by-products, such as pyruvate by-products (e.g., alanine, and/or valine), TCA derived by-products, acetate and/or ethanol, for enhancing carbon flux through acetyl-CoA to generate a non-naturally occurring microbial organism that further produces any pathway intermediate or product derived from acetyl-CoA, as desired. As disclosed herein, such a microbial organism that produces an intermediate can be used in combination with another microbial organism expressing downstream pathway enzymes to produce a desired product. However, it is understood that a non-naturally occurring microbial organism that produces an intermediate of an acetyl-CoA derived product, such as an intermediate of a 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA biosynthesis pathway can be utilized to produce the intermediate as a desired product.

The invention is described herein with general reference to reducing by-products of the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well-known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms of the invention can be produced by introducing expressible nucleic acids encoding one or more of the enzymes or proteins of the acetaldehyde recycling loop, and/or attenuating an acetolactate synthase, to reduce by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, and related products derived therefrom. In addition, in some embodiments, the non-naturally occurring microbial organisms of the invention can be produced by further introducing one or more of the enzymes or proteins participating in, for example, one or more 1,3-butanediol (1,3-BDO), methyl methacrylate (MMA), (3R)-hydroxybutyl (3R)-hydroxybutyrate, or any other acetyl-CoA derived product biosynthesis pathways.

Depending on the host microbial organism chosen for biosynthesis, nucleic acids for some or all of a particular biosynthetic pathway that converts acetyl-CoA into a desired product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve biosynthesis of 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. E. coli is a particularly useful host organisms since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the biosynthetic pathway that utilizes acetyl-CoA for production of a desired product such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, and the constituents of a selected host microbial organism, the non-naturally occurring microbial organisms of the invention can include at least one exogenously expressed 1,3-BDO, MMA, (3R)-hydroxybutyl (3R)-hydroxybutyrate, or any other acetyl-CoA derived product pathway-encoding nucleic acid and up to all encoding nucleic acids for one or more 1,3-BDO, MMA, (3R)-hydroxybutyl (3R)-hydroxybutyrate, or any other acetyl-CoA derived product biosynthetic pathways. For example, 1,3-BDO, MMA, or (3R)-hydroxybutyl (3R)-hydroxybutyrate biosynthesis can be established in a host deficient in a pathway enzyme or protein through exogenous expression of the corresponding encoding nucleic acid. In a host deficient in all enzymes or proteins of a 1,3-BDO, MMA, (3R)-hydroxybutyl (3R)-hydroxybutyrate, or any other acetyl-CoA derived product pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of 1,3-BDO, (3R)-hydroxybutyl (3R)-hydroxybutyrate, MMA, or any other acetyl-CoA derived product can be included in a host expressing one or more enzymes or proteins of an acetaldehyde recycling loop and/or having an attenuated for acetolactate synthase for reducing by-products, such as pyruvate by-products (e.g., alanine, and/or valine), TCA derived by-products, acetate and/or ethanol.

Given the teachings and guidance provided herein, those skilled in the art will understand that the number of encoding nucleic acids to introduce in an expressible form will, at least, parallel the pathway deficiencies of the selected host microbial organism having reduced by-products. For example, a non-naturally occurring microbial organism of the invention can have one, or two nucleic acids encoding the enzymes or proteins constituting an acetaldehyde recycling loop pathway disclosed herein for reducing by-products, such as acetate and/or ethanol, for enhancing carbon flux through acetyl-CoA. The enhanced carbon flux through acetyl-CoA can thereby increase acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the non-naturally occurring microbial organisms also can include other genetic modifications that facilitate or optimize the biosynthesis of acetyl-CoA derived products, such as 1,3-BDO, MMA, (3R)-hydroxybutyl (3R)-hydroxybutyrate, or any other acetyl-CoA derived product, or that confer other useful functions onto the host microbial organism. One such other functionality can include, for example, augmentation of the synthesis of one or more of the acetyl-CoA derived product pathway precursors such as a precursor of 1,3-BDO (e.g., 3HB-CoA), MMA (e.g., MAA-CoA), (3R)-hydroxybutyl (3R)-hydroxybutyrate (e.g., acetoacetyl-CoA), or a precursor of any other acetyl-CoA derived product.

Therefore, a non-naturally occurring microbial organism of the invention having reduced by-products can have one or two nucleic acids encoding the enzymes or proteins constituting an acetaldehyde recycling loop pathway disclosed herein. In some embodiments, a non-naturally occurring microbial organism of the invention having reduced by-products can further have can have one, two, three, four, five, six, seven or eight up to all nucleic acids encoding the enzymes or proteins constituting a reaction biosynthetic pathway disclosed herein. In some embodiments, the non-naturally occurring microbial organisms also can include other genetic modifications that facilitate or optimize the biosynthesis of acetyl-CoA derived products, such as 1,3-BDO, MMA, (3R)-hydroxybutyl (3R)-hydroxybutyrate, or any other acetyl-CoA derived product, or that confer other useful functions onto the host microbial organism. By way of example, one such other functionality can include, for example, augmentation of the synthesis of one or more of the 1,3-BDO pathway precursors such as 3HB-CoA.

Generally, a host microbial organism is selected such that it produces the precursor of an acetyl-CoA dependent pathway, either as a naturally produced molecule or as an engineered product that either provides de novo production of a desired precursor or increased production of a precursor naturally produced by the host microbial organism. For example, pyruvate is produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a pathway for increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In some embodiments, a non-naturally occurring microbial organism of the invention having reduced by-products, such as pyruvate by-products (e.g., alanine, and/or valine), TCA derived by-products, acetate and/or ethanol, is generated from a host that contains the enzymatic capability to synthesize an acetyl-CoA derived product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In this specific embodiment it can be useful to increase the synthesis or accumulation of an acetyl-CoA derived product pathway product to, for example, drive 1,3-BDO, MMA, (3R)-hydroxybutyl (3R)-hydroxybutyrate, or any other acetyl-CoA derived product pathway reactions toward production of 1,3-BDO, MMA, (3R)-hydroxybutyl (3R)-hydroxybutyrate, or any other acetyl-CoA derived product, respectively. Increased synthesis or accumulation can be accomplished by, for example, overexpression of nucleic acids encoding enzymes or proteins involved in pathways for producing acetyl-CoA derived products.

Over expression of the enzyme or enzymes and/or protein or proteins disclosed herein that are capable of decreasing by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, having reduced by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA through overexpression of one, two, three, four, five, six, seven, or eight, depending on the number of enzymes in the pathway, that is, up to all nucleic acids encoding enzymes or proteins disclosed herein that can reduce by-products, as well as increase, for example, 1,3-BDO, MMA, (3R)-hydroxybutyl (3R)-hydroxybutyrate, or any other acetyl-CoA derived product biosynthetic pathway enzymes or proteins. In addition, a non-naturally occurring organism can be generated by mutagenesis of an endogenous gene that results in an increase in activity of an enzyme disclosed herein that can decrease by-products, as well as increase, for example, a 1,3-BDO, MMA, (3R)-hydroxybutyl (3R)-hydroxybutyrate, or any other acetyl-CoA derived product.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments, such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention having reduced by-products, such as pyruvate by-products (e.g., alanine, and/or valine), TCA derived by-products, acetate and/or ethanol, for enhancing carbon flux through acetyl-CoA. The nucleic acids can be introduced so as to confer, for example, a microbial organism having an attenuated acetolactate synthase, and/or an acetaldehyde recycling loop. In some embodiments, the microbial organism having reduced by-products can further include nucleic acids introduced so as to confer, for example, a microbial organism having a biosynthetic pathway for production of 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. Alternatively, encoding nucleic acids can be introduced to produce an intermediate microbial organism having the biosynthetic capability to catalyze some of the required reactions to confer biosynthetic capability of 1,3-BDO, MMA, (3R)-hydroxybutyl (3R)-hydroxybutyrate, or any other acetyl-CoA derived product. For example, a non-naturally occurring microbial organism having reduced by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA can comprise at least two exogenous nucleic acids encoding desired enzymes or proteins, such as the combination of an attenuated acetolactate synthase, and an acetyl-CoA synthase; and attenuated acetolactate synthase and an aldehyde dehydrogenase; an attenuated acetolactate synthase and an aldehyde dehydrogenase; an aldehyde dehydrogenase and an acetyl-CoA synthase, and the like. Thus, it is understood that any combination of two or more enzymes or proteins of a biosynthetic pathway can be included in a non-naturally occurring microbial organism of the invention. Similarly, it is understood that all three enzymes or proteins of a biosynthetic pathway can be included in a non-naturally occurring microbial organism of the invention, for example, an attenuated acetolactate synthase, an acetyl-CoA synthase and an aldehyde dehydrogenase.

In addition to the biosynthesis of, for example, 1,3-BDO, (3R)-hydroxybutyl (3R)-hydroxybutyrate, MMA, or any other acetyl-CoA derived product as described herein, the non-naturally occurring microbial organisms having decreased by-products, such as pyruvate by-products (e.g., alanine, and/or valine), TCA derived by-products, acetate and/or ethanol, for enhancing carbon flux through acetyl-CoA and methods of the invention for decreasing such by-products can also be utilized in various combinations with each other and with other microbial organisms and methods well known in the art to achieve product biosynthesis by other routes. For example, one alternative to produce, for example, 1,3-BDO, (3R)-hydroxybutyl (3R)-hydroxybutyrate, MMA, or any other acetyl-CoA derived product in a non-naturally occurring microbial organism having decreased by-products, such as pyruvate by-products (e.g., alanine, and/or valine), TCA derived by-products, acetate and/or ethanol, for enhancing carbon flux through acetyl-CoA other than use of the, for example, 1,3-BDO, (3R)-hydroxybutyl (3R)-hydroxybutyrate, MMA, or other acetyl-CoA derived product producers is through addition of another microbial organism capable of converting, for example, a 1,3-BDO, (3R)-hydroxybutyl (3R)-hydroxybutyrate, MMA, or other acetyl-CoA derived product biosynthesis pathway intermediate to, for example, 1,3-BDO, (3R)-hydroxybutyl (3R)-hydroxybutyrate, MMA, or other acetyl-CoA derived product, respectively.

One such procedure includes, for example, the fermentation of a microbial organism that produces a 1,3-BDO, (3R)-hydroxybutyl (3R)-hydroxybutyrate, MMA, or other acetyl-CoA derived product biosynthesis pathway intermediate. The 1,3-BDO, (3R)-hydroxybutyl (3R)-hydroxybutyrate, MMA, or other acetyl-CoA derived product biosynthesis pathway intermediate can then be used as a substrate for a second non-naturally occurring microbial, that converts the 1,3-BDO, (3R)-hydroxybutyl (3R)-hydroxybutyrate, MMA, or other acetyl-CoA derived product biosynthesis pathway intermediate to 1,3-BDO, (3R)-hydroxybutyl (3R)-hydroxybutyrate, MMA, or other acetyl-CoA derived product, respectively. The 1,3-BDO, (3R)-hydroxybutyl (3R)-hydroxybutyrate, MMA, or other acetyl-CoA derived product biosynthesis pathway intermediate can be added directly to another culture of the second non-naturally occurring microbial organism or the original culture of the 1,3-BDO, (3R)-hydroxybutyl (3R)-hydroxybutyrate, MMA, or other acetyl-CoA derived product biosynthesis pathway intermediate producers can be depleted of these microbial organisms by, for example, cell separation, and then subsequent addition of the second non-naturally occurring microbial organism to the fermentation broth can be utilized to produce the final product without intermediate purification steps.

In other embodiments, the non-naturally occurring microbial organisms disclosed herein having reduced by-products, such as pyruvate by-products (e.g., alanine, and/or valine), TCA derived by-products, acetate and/or ethanol, for enhancing carbon flux through acetyl-CoA and methods of the invention for reducing such by-products can be assembled in a wide variety of subpathways to achieve biosynthesis of, for example, 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of 1,3-BDO, (3R)-hydroxybutyl (3R)-hydroxybutyrate, MMA, or any other acetyl-CoA derived product, can be accomplished by constructing a microbial organism that contains biosynthetic pathways for conversion of one pathway intermediate to another pathway intermediate or the product, and one or more of the microbial organisms that perform conversion of one pathway intermediate to another pathway intermediate or the product via an acetyl-CoA derived pathway can be constructed to have reduced by-products, such as pyruvate by-products (e.g., alanine, and/or valine), TCA derived by-products, acetate and/or ethanol, for enhancing carbon flux through acetyl-CoA and thereby increasing acetyl-CoA derived products. Alternatively, the production of a desired acetyl-CoA derived product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, 1,3-BDO, (3R)-hydroxybutyl (3R)-hydroxybutyrate, MMA, or an amino acid, can also be biosynthetically produced from one or more microbial organisms having enhanced carbon flux through acetyl-CoA through co-culture or co-fermentation using two organisms in the same vessel, where the first microbial organism produces an intermediate, such as an 1,3-BDO intermediate, and the second microbial organism converts the intermediate to 1,3-BDO.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

Similarly, it is understood by those skilled in the art that a host organism can be selected based on desired characteristics for introduction of one or more gene disruptions to increase production of 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods of the invention can be applied to any suitable host microorganism to identify the cognate metabolic alterations needed to construct an organism in a species of interest that will increase biosynthesis of 1,3-BDO, MMA, (3R)-hydroxybutyl (3R)-hydroxybutyrate, or any other acetyl-CoA derived product. In a particular embodiment, the increased production couples biosynthesis of, for example, 1,3-BDO, MMA, or (3R)-hydroxybutyl (3R)-hydroxybutyrate, and can obligatorily couple production of for example, 1,3-BDO, MMA, or (3R)-hydroxybutyl (3R)-hydroxybutyrate, to growth of the organism if desired and as disclosed herein.

Sources of encoding nucleic acids for an acetaldehyde recycling loop, or a pathway enzyme or protein that uses acetyl-CoA as a substrate can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, as well as other exemplary species disclosed herein or available as source organisms for corresponding genes. Exemplary species for such sources include, for example, Escherichia coli, Escherichia fergusonii, Methanocaldococcus jannaschii, Leptospira interrrogans, Geobacter sulfurreducens, Chloroflexus aurantiacus, Roseiflexus sp. RS-1, Chloroflexus aggregans, Achromobacter xylosoxydans, Clostrdia species, including Clostridium kluyveri, Clostridium symbiosum, Clostridium acetobutylicum, Clostridium saccharoperbutylacetonicum, Clostridium ljungdahlii, Trichomonas vaginalis G3, Trypanosoma brucei, Acidaminococcus fermentans, Fusobacterium species, including Fusobacterium nucleatum, Fusobacterium mortiferum, Corynebacterium glutamicum, Rattus norvegicus, Homo sapiens, Saccharomyces species, including Saccharomyces cerevisiae, Apsergillus species, including Aspergillus terreus, Aspergillus oryzae, Aspergillus niger, Gibberella zeae, Pichia stipitis, Mycobacterium species, including Mycobacterium smegmatis, Mycobacterium avium, including subsp. pratuberculosis, Salinispora arenicola Pseudomonas species, including Pseudomonas sp. CF600, Pseudomonas putida, Pseudomonas fluorescens, Pseudomonas aeruginosa, Ralstonia species, including Ralstonia eutropha, Ralstonia eutropha JMP 134, Ralstonia eutropha H16, Ralstonia pickettii, Lactobacillus plantarum, Klebsiella oxytoca, Bacillus species, including Bacillus methanolicus, Bacillus subtilis, Bacillus pumilus, Bacillus megaterium, Pedicoccus pentosaceus, Chlorofexus species, including Chloroflexus aurantiacus, Chloroflexus aggregans, Rhodobacter sphaeroides, Methanocaldococcus jannaschii, Leptospira interrrogans, Candida maltosa, Salmonella species, including Salmonella enterica serovar Typhimurium, Shewanella species, including Shewanella oneidensis, Shewanella sp. MR-4, Alcaligenes faecalis, Geobacillus stearothermophilus, Serratia marcescens, Vibrio cholerae, Eubacterium barkeri, Bacteroides capillosus, Archaeoglobus fulgidus, Archaeoglobus fulgidus, Haloarcula marismortui, Pyrobaculum aerophilum str. IM2, Rhizobium species, including Rhizobium leguminosarum as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite activity to decrease by-products, such as pyruvate by-products (e.g., alanine, and/or valine), TCA derived by-products, acetate and/or ethanol, for enhancing carbon flux through acetyl-CoA, along with genes encoding 1,3-BDO, (3R)-hydroxybutyl (3R)-hydroxybutyrate, MMA, or other acetyl-CoA derived product biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing reduced by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative biosynthetic pathway for production of 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, exists in an unrelated species, biosynthesis of 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

Methods for constructing and testing the expression levels of a non-naturally occurring organism having reduce by-products, such as pyruvate by-products (e.g., alanine, and/or valine), TCA derived by-products, acetate and/or ethanol, for enhancing carbon flux through acetyl-CoA can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for reducing by-products, such as pyruvate by-products (e.g., alanine, and/or valine), TCA derived by-products, acetate and/or ethanol, to enhance carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more exogenous nucleic acids each encoding an enzyme expressed in a sufficient amount to decrease by-products, pyruvate by-products, acetate and/or ethanol, for enhancing carbon flux through acetyl-CoA, as exemplified herein operably linked to expression control sequences functional in the host organism. In some embodiments, an expression vector or vectors can be further constructed to include one or more exogenous nucleic acids each encoding an enzyme expressed in a sufficient amount to increase production of an acetyl-CoA derived product such as, for example, 1,3-BDO, (3R)-hydroxybutyl (3R)-hydroxybutyrate, MMA, or other acetyl-CoA derived product biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

Suitable purification and/or assays to test for the production of by-products, such as pyruvate by-products (e.g., alanine, and/or valine), TCA derived by-products, acetate and/or ethanol, acetyl-CoA, or acetyl-CoA derived products such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA can be performed using well known methods. Suitable replicates such as triplicate cultures can be grown for each engineered strain to be tested. For example, product and by-product formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. By-products and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The production of acetyl-CoA derived compounds, such as for example, 1,3-BDO, (3R)-hydroxybutyl (3R)-hydroxybutyrate, MMA, or any other acetyl-CoA derived product including, but not limited to, 4OH2B, 3HB, adipate, caprolactam, 6-ACA, HMDA, or MAA, can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms having decreased by-products, such as pyruvate by-products (e.g., alanine, and/or valine), TCA derived by-products, acetate and/or ethanol, for enhancing carbon flux through acetyl-CoA, as described herein, can be cultured to produce and/or secrete the biosynthetic acetyl-CoA derived products of the invention. For example, the acetyl-CoA derived product producers can be cultured for the biosynthetic production of a desired acetyl-CoA derived product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

For the production of the desired acetyl-CoA derived product, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention having reduced by-products, such as pyruvate by-products (e.g., alanine, and/or valine), TCA derived by-products, acetate and/or ethanol, for the production of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

In addition to renewable feedstocks such as those exemplified above, the microbial organisms of the invention having decreased by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA can also be modified for growth on syngas as its source of carbon. In this specific embodiment, one or more proteins or enzymes are expressed in the acetyl-CoA derived product producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+nADP+nPi→CH₃COOH+2H₂O+nATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to reduce generation of unwanted by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse) tricarboxylic acid cycle is and/or hydrogenase activities can also be used for the conversion of CO, CO2 and/or H2 to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H2 by carbon monoxide dehydrogenase and hydrogenase are utilized to fix CO2 via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to, for example, precursors of acetyl-CoA derived products, such as precursors of 1,3-BDO, MMA, (3R)-hydroxybutyl (3R)-hydroxybutyrate, or any other acetyl-CoA derived product, glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate, for example, a 1,3-BDO pathway, a (3R)-hydroxybutyl (3R)-hydroxybutyrate pathway, a MMA pathway, or any other biosynthesis pathway that utilizes acetyl-CoA as a substrate in its pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains the complete reductive TCA pathway will confer syngas utilization ability.

Accordingly, given the teachings and guidance provided herein, those skilled in the art will understand that a non-naturally occurring microbial organism having decreased unwanted by-products, such as pyruvate by-products (e.g., alanine, and/or valine), TCA derived by-products, acetate and/or ethanol, for enhancing carbon flux through acetyl-CoA, can be produced that secretes the biosynthesized compounds of the invention when grown on a carbon source such as a carbohydrate. Such compounds include, for example, an acetyl-CoA derived product, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, and any of the intermediate metabolites therefrom. All that is required is to engineer in one or more of the required enzyme or protein activities to achieve biosynthesis of the desired compound or intermediate including, for example, inclusion of some or all of the acetyl-CoA derived product biosynthetic pathways. Accordingly, the invention provides a non-naturally occurring microbial organism that produces and/or secretes, an acetyl-CoA derived product, such as 1,3-BDO, MMA, (3R)-hydroxybutyl (3R)-hydroxybutyrate, or any other acetyl-CoA derived product when grown on a carbohydrate or other carbon source and produces and/or secretes any of the intermediate metabolites in the acetyl-CoA derived product pathway when grown on a carbohydrate or other carbon source. The acetyl-CoA derived product producing microbial organisms of the invention can initiate synthesis from an intermediate.

In some embodiments, the non-naturally occurring microbial organisms of the invention are constructed using methods well known in the art as exemplified herein to exogenously express at least one nucleic acid encoding an enzyme or protein in sufficient amounts to reduce by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In other embodiments, the non-naturally occurring microbial organisms of the invention are constructed using methods well known in the art as exemplified herein to attenuate the activity of an enzyme or protein in sufficient amounts to reduce by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA.

It is understood that the microbial organisms of the invention are cultured under conditions sufficient to produce acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. Following the teachings and guidance provided herein, the non-naturally occurring microbial organisms of the invention can achieve biosynthesis of an acetyl-CoA derived product, such as 1,3-BDO, MMA, (3R)-hydroxybutyl (3R)-hydroxybutyrate, or any other acetyl-CoA derived product, resulting in intracellular concentrations between about 0.1-200 mM or more. Generally, the intracellular concentration of the acetyl-CoA derived product, such as 1,3-BDO, (3R)-hydroxybutyl (3R)-hydroxybutyrate, or MMA, is between about 30-300 mM, particularly between about 50-200 mM and more particularly between about 70-150 mM, including about 70 mM, 80 mM, 90 mM, 100 mM, 110 mM, 120 mM, 130 mM, 140 mM, 150 mM or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the products of acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA can synthesize the desired acetyl-CoA derived product at intracellular concentrations of 5-10 mM or more as well as all other concentrations exemplified herein. It is understood that, even though the above description refers to intracellular concentrations, the acetyl-CoA derived product producing microbial organisms can produce the acetyl-CoA derived product, such as 1,3-BDO, MMA, (3R)-hydroxybutyl (3R)-hydroxybutyrate, or any other acetyl-CoA derived product, intracellularly and/or secrete the product into the culture medium.

In addition to the culturing and fermentation conditions disclosed herein, growth condition for achieving biosynthesis of, for example, 1,3-BDO, MMA, (3R)-hydroxybutyl (3R)-hydroxybutyrate, or any other acetyl-CoA derived product can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethyl slfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of, for example, 1,3-BDO, MMA, (3R)-hydroxybutyl (3R)-hydroxybutyrate, or any other acetyl-CoA derived product includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N2/CO2 mixture or other suitable non-oxygen gas or gases.

The culture conditions described herein can be scaled up and grown continuously for manufacturing of, for example, 1,3-BDO, MMA, (3R)-hydroxybutyl (3R)-hydroxybutyrate, or any other acetyl-CoA derived product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of, for example, 1,3-BDO, MMA, (3R)-hydroxybutyl (3R)-hydroxybutyrate, or any other acetyl-CoA derived product. Generally, and as with non-continuous culture procedures, the continuous and/or near-continuous production of, for example, 1,3-BDO, MMA, (3R)-hydroxybutyl (3R)-hydroxybutyrate, or any other acetyl-CoA derived product will include culturing a non-naturally occurring 1,3-BDO, MMA, (3R)-hydroxybutyl (3R)-hydroxybutyrate, or any other acetyl-CoA derived product producing organism of the invention in sufficient nutrients and medium to sustain and/or nearly sustain growth in an exponential phase. Continuous culture under such conditions can be include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of, for example, 1,3-BDO, MMA, (3R)-hydroxybutyl (3R)-hydroxybutyrate, or any other acetyl-CoA derived product can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 1,3-BDO, MMA, (3R)-hydroxybutyl (3R)-hydroxybutyrate, or any other acetyl-CoA derived product producers of the invention for continuous production of substantial quantities of 1,3-BDO, MMA, (3R)-hydroxybutyl (3R)-hydroxybutyrate, or any other acetyl-CoA derived product, the 1,3-BDO, MMA, (3R)-hydroxybutyl (3R)-hydroxybutyrate, or any other acetyl-CoA derived product producers also can be, for example, simultaneously subjected to chemical synthesis procedures to convert the product to other compounds or the product can be separated from the fermentation culture and sequentially subjected to chemical conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of 1,3-BDO, MMA, (3R)-hydroxybutyl (3R)-hydroxybutyrate, or any other acetyl-CoA derived product.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory by-product of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007, which are incorporated by reference in their entirety.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

As provided herein, the invention also provides non naturally occurring microbial organisms having genetic alterations such as gene disruptions that decrease the production of by-products, such as pyruvate by-products (e.g., alanine, and/or valine), TCA derived by-products, acetate and/or ethanol, to enhance carbon flux through acetyl-CoA and thereby increase acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. In some embodiments, the non-naturally occurring microbial organism of the present invention includes a deletion of acetolactate synthase. In some embodiments, the deletion of acetolactate synthase includes deletion of ilvG.

Given the teachings and guidance provided herein, those skilled in the art will understand that to introduce a metabolic alteration such as disruption of an enzymatic reaction, it is necessary to disrupt the catalytic activity of the one or more enzymes involved in the reaction. Alternatively, a metabolic alteration can include disruption of expression of a regulatory protein or cofactor necessary for enzyme activity or maximal activity. Disruption can occur by a variety of methods including, for example, deletion of an encoding gene or incorporation of a genetic alteration in one or more of the encoding gene sequences. The encoding genes targeted for disruption can be one, some, or all of the genes encoding enzymes involved in the catalytic activity. For example, where a single enzyme is involved in a targeted catalytic activity, disruption can occur by a genetic alteration that reduces or eliminates the catalytic activity of the encoded gene product. Similarly, where the single enzyme is multimeric, including heteromeric, disruption can occur by a genetic alteration that reduces or destroys the function of one or all subunits of the encoded gene products. Destruction of activity can be accomplished by loss of the binding activity of one or more subunits required to form an active complex, by destruction of the catalytic subunit of the multimeric complex or by both. Other functions of multimeric protein association and activity also can be targeted in order to disrupt a metabolic reaction of the invention. Such other functions are well known to those skilled in the art. Similarly, a target enzyme activity can be reduced or eliminated by disrupting expression of a protein or enzyme that modifies and/or activates the target enzyme, for example, a molecule required to convert an apoenzyme to a holoenzyme. Further, some or all of the functions of a single polypeptide or multimeric complex can be disrupted according to the invention in order to reduce or abolish the catalytic activity of one or more enzymes involved in a reaction or metabolic modification of the invention. Similarly, some or all of enzymes involved in a reaction or metabolic modification of the invention can be disrupted so long as the targeted reaction is reduced or eliminated.

Given the teachings and guidance provided herein, those skilled in the art also will understand that an enzymatic reaction can be disrupted by reducing or eliminating reactions encoded by a common gene and/or by one or more orthologs of that gene exhibiting similar or substantially the same activity. Reduction of both the common gene and all orthologs can lead to complete abolishment of any catalytic activity of a targeted reaction. However, disruption of either the common gene or one or more orthologs can lead to a reduction in the catalytic activity of the targeted reaction sufficient to promote coupling of growth to product biosynthesis. Exemplified herein are both the common genes encoding catalytic activities for a variety of metabolic modifications as well as their orthologs. Those skilled in the art will understand that disruption of some or all of the genes encoding an enzyme of a targeted metabolic reaction can be practiced in the methods of the invention and incorporated into the non-naturally occurring microbial organisms of the invention in order to achieve the decreased production of by-products, such as pyruvate by-products (e.g., alanine, and/or valine), TCA derived by-products, acetate and/or ethanol, to enhance carbon flux through acetyl-CoA.

In some embodiments, microaerobic designs can be used based on the growth-coupled formation of the desired product. To examine this, production cones can be constructed for each strategy by first maximizing and, subsequently minimizing the product yields at different rates of biomass formation feasible in the network. If the rightmost boundary of all possible phenotypes of the mutant network is a single point, it implies that there is a unique optimum yield of the product at the maximum biomass formation rate possible in the network. In other cases, the rightmost boundary of the feasible phenotypes is a vertical line, indicating that at the point of maximum biomass the network can make any amount of the product in the calculated range, including the lowest amount at the bottommost point of the vertical line. Such designs are given a low priority.

In some embodiments, the gene disruption can include a complete gene deletion. In some embodiments other methods to disrupt a gene include, for example, frameshifting by omission or addition of oligonucleotides or by mutations that render the gene inoperable. One skilled in the art will recognize the advantages of gene deletions, however, because of the stability it confers to the non-naturally occurring organism from reverting to a parental phenotype in which the gene disruption has not occurred. In particular, the gene disruptions are selected from the gene sets as disclosed herein.

Once computational predictions are made of gene sets for disruption to reduce by-products, such as pyruvate by-products (e.g., alanine, and/or valine), TCA derived by-products, acetate and/or ethanol, for enhancing carbon flux through acetyl-CoA and thereby increasing acetyl-CoA derived products, the strains can be constructed, evolved, and tested. Gene disruptions, including gene deletions, are introduced into host organism by methods well known in the art. A particularly useful method for gene disruption is by homologous recombination, as disclosed herein.

The engineered strains can be characterized by measuring the growth rate, the substrate uptake rate, and/or the product/by-product secretion rate. Cultures can be grown and used as inoculum for a fresh batch culture for which measurements are taken during exponential growth. The growth rate can be determined by measuring optical density using a spectrophotometer (A600). Concentrations of glucose and other organic acid by-products in the culture supernatant can be determined by well-known methods such as HPLC, GC-MS or other well-known analytical methods suitable for the analysis of the desired product, as disclosed herein, and used to calculate uptake and secretion rates.

Strains containing gene disruptions can exhibit suboptimal growth rates until their metabolic networks have adjusted to their missing functionalities. To assist in this adjustment, the strains can be adaptively evolved. By subjecting the strains to adaptive evolution, cellular growth rate becomes the primary selection pressure and the mutant cells are compelled to reallocate their metabolic fluxes in order to enhance their rates of growth. This reprogramming of metabolism has been recently demonstrated for several E. coli mutants that had been adaptively evolved on various substrates to reach the growth rates predicted a priori by an in silico model (Fong and Palsson, Nat. Genet. 36:1056-1058 (2004)). The growth improvements brought about by adaptive evolution can be accompanied by enhanced rates of [INSERT PRODUCT] production. The strains are generally adaptively evolved in replicate, running in parallel, to account for differences in the evolutionary patterns that can be exhibited by a host organism (Fong and Palsson, Nat. Genet. 36:1056-1058 (2004); Fong et al., J. Bacteriol. 185:6400-6408 (2003); Ibarra et al., Nature 420:186-189 (2002)) that could potentially result in one strain having superior production qualities over the others. Evolutions can be run for a period of time, typically 2-6 weeks, depending upon the rate of growth improvement attained. In general, evolutions are stopped once a stable phenotype is obtained.

Following the adaptive evolution process, the new strains are characterized again by measuring the growth rate, the substrate uptake rate, and the product/by-product secretion rate. These results are compared to the theoretical predictions by plotting actual growth and production yields alongside the production envelopes from metabolic modeling. The most successful design/evolution combinations are chosen to pursue further, and are characterized in lab-scale batch and continuous fermentations. The growth-coupled biochemical production concept behind the methods disclosed herein such as OptKnock approach should also result in the generation of genetically stable overproducers. Thus, the cultures are maintained in continuous mode for an extended period of time, for example, one month or more, to evaluate long-term stability. Periodic samples can be taken to ensure that yield and productivity are maintained.

Adaptive evolution is a powerful technique that can be used to increase growth rates of mutant or engineered microbial strains, or of wild-type strains growing under unnatural environmental conditions. It is especially useful for strains designed via methods such as OptKnock, which results in growth-coupled product formation. Therefore, evolution toward optimal growing strains will indirectly optimize production as well. Unique strains of E. coli K-12 MG1655 were created through gene knockouts and adaptive evolution. (Fong and Palsson, Nat. Genet. 36:1056-1058 (2004)). In this work, all adaptive evolutionary cultures were maintained in prolonged exponential growth by serial passage of batch cultures into fresh medium before the stationary phase was reached, thus rendering growth rate as the primary selection pressure. Knockout strains were constructed and evolved on minimal medium supplemented with different carbon substrates (four for each knockout strain). Evolution cultures were carried out in duplicate or triplicate, giving a total of 50 evolution knockout strains. The evolution cultures were maintained in exponential growth until a stable growth rate was reached. The computational predictions were accurate (within 10%) at predicting the post-evolution growth rate of the knockout strains in 38 out of the 50 cases examined. Furthermore, a combination of OptKnock design with adaptive evolution has led to improved lactic acid production strains. (Fong et al., Biotechnol. Bioeng. 91:643-648 (2005)). Similar methods can be applied to the strains disclosed herein and applied to various host strains.

There are a number of developed technologies for carrying out adaptive evolution. Exemplary methods are disclosed herein. In some embodiments, optimization of a non-naturally occurring organism of the present invention having reduced by-products, such as pyruvate by-products (e.g., alanine, and/or valine), TCA derived by-products, acetate and/or ethanol, includes utilizing adaptive evolution techniques for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, to increase production and/or stability of the producing strain.

Serial culture involves repetitive transfer of a small volume of grown culture to a much larger vessel containing fresh growth medium. When the cultured organisms have grown to saturation in the new vessel, the process is repeated. This method has been used to achieve the longest demonstrations of sustained culture in the literature (Lenski and Travisano, Proc. Natl. Acad. Sci. USA 91:6808-6814 (1994)) in experiments which clearly demonstrated consistent improvement in reproductive rate over a period of years. Typically, transfer of cultures is usually performed during exponential phase, so each day the transfer volume is precisely calculated to maintain exponential growth through the next 24 hour period. Manual serial dilution is inexpensive and easy to parallelize.

In continuous culture the growth of cells in a chemostat represents an extreme case of dilution in which a very high fraction of the cell population remains. As a culture grows and becomes saturated, a small proportion of the grown culture is replaced with fresh media, allowing the culture to continually grow at close to its maximum population size. Chemostats have been used to demonstrate short periods of rapid improvement in reproductive rate (Dykhuizen, Methods Enzymol. 613-631 (1993)). The potential usefulness of these devices was recognized, but traditional chemostats were unable to sustain long periods of selection for increased reproduction rate, due to the unintended selection of dilution-resistant (static) variants. These variants are able to resist dilution by adhering to the surface of the chemostat, and by doing so, outcompete less adherent individuals, including those that have higher reproductive rates, thus obviating the intended purpose of the device (Chao and Ramsdell, J. Gen. Microbiol 20:132-138 (1985)). One possible way to overcome this drawback is the implementation of a device with two growth chambers, which periodically undergo transient phases of sterilization, as described previously (Marliere and Mutzel, U.S. Pat. No. 6,686,194).

Evolugator™ is a continuous culture device developed by Evolugate, LLC (Gainesville, Fla.) and exhibits significant time and effort savings over traditional evolution techniques (de Crecy et al., Appl. Microbiol. Biotechnol. 77:489-496 (2007)). The cells are maintained in prolonged exponential growth by the serial passage of batch cultures into fresh medium before the stationary phase is attained. By automating optical density measurement and liquid handling, the Evolugator™ can perform serial transfer at high rates using large culture volumes, thus approaching the efficiency of a chemostat in evolution of cell fitness. For example, a mutant of Acinetobacter sp ADP1 deficient in a component of the translation apparatus, and having severely hampered growth, was evolved in 200 generations to 80% of the wild-type growth rate. However, in contrast to the chemostat which maintains cells in a single vessel, the machine operates by moving from one “reactor” to the next in subdivided regions of a spool of tubing, thus eliminating any selection for wall-growth. The transfer volume is adjustable, and normally set to about 50%. A drawback to this device is that it is large and costly, thus running large numbers of evolutions in parallel is not practical. Furthermore, gas addition is not well regulated, and strict anaerobic conditions are not maintained with the current device configuration. Nevertheless, this is an alternative method to adaptively evolve a production strain.

As disclosed herein, a nucleic acid encoding a desired activity of an enzyme that decreases unwanted by-products, such as pyruvate by-products (e.g., alanine, and/or valine), TCA derived by-products, acetate and/or ethanol, for enhancing carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA, can be introduced into a host organism. In some cases, it can be desirable to modify an activity of an enzyme or protein that decreases unwanted by-products or protein to increase carbon flux through acetyl-CoA and thereby increasing acetyl-CoA derived products, such as 1,3-BDO, MMA, (3R)-hydroxybutyl (3R)-hydroxybutyrate, or any other acetyl-CoA derived product including, but not limited to, 4OH2B, 3HB, adipate, caprolactam, 6-ACA, HMDA, or MAA. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >104). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol.Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of an enzyme or protein that decreases unwanted by-products, such as pyruvate by-products (e.g., alanine, and/or valine), TCA derived by-products, acetate and/or ethanol, for enhancing carbon flux through acetyl-CoA. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol.Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)). Any of the aforementioned methods for mutagenesis can be used alone or in any combination. Additionally, any one or combination of the directed evolution methods can be used in conjunction with adaptive evolution techniques, as described herein.

### 6. EXAMPLES

### Example I

### Attenuation of Acetolactate Synthase Improved Titer, Rate, and Yield of 1,3-butanediol (1,3-BDO)

The following example demonstrates that attenuation of the valine biosynthesis enzyme, acetolactate synthase (ilvG), improved titer, rate, and yield of 1,3-butanediol (1,3-BDO). The E.Coli strain L16375, which expresses nadK and pntAB, and the E. Coli strains L16410 and L16411, which express nadK and pntAB and have a deletion of the ilvGM operon, were cultured and production of 1,3-BDO was measured.

Cultivation of three different bacterial strains (L16410, L16411, and L16375) was performed using standard techniques with a flow rate of 300 standard cubic centimeters per minute (sccm) at 100% air in 2.5 mL, 2.0 mL, or 1.5 mL of culture media with a power of 48 WP. The cultures were incubated at 400 rpm for 24 hours, with a starting OD of 0.4.

TABLE 1

| Strain | Detail |
|---|---|
| L16375 | ECh-10481 (ECh-10437 p108-pntAB-p115-nadK) |
| L16410 | ECh-10488 (ECh-10452, ΔilvGM, p115-nadK-pntAB) |
| L16411 | ECh-10489 (ECh-10481, ΔilvGM, p108-pntAB-p115-nadK) |

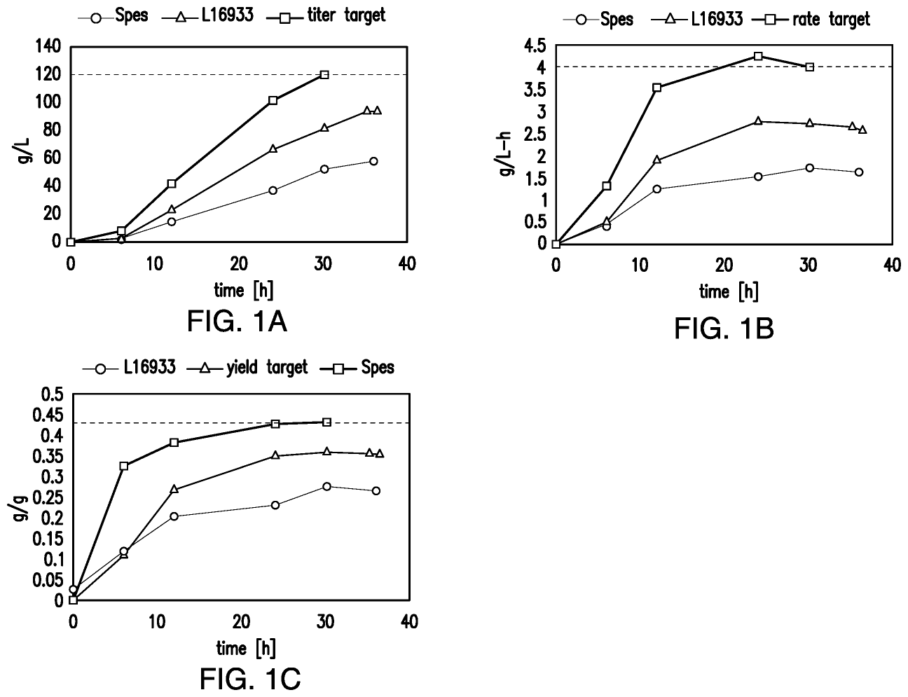

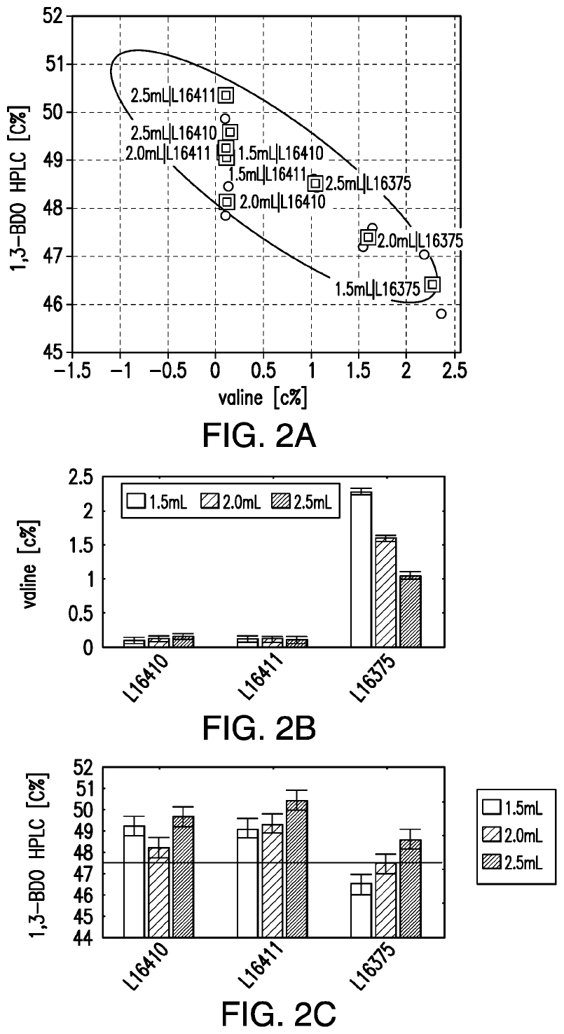

The results indicated that the ilvGM deletion strains (L16410 and L16411) performed better than control (L16375) with regard to specific 1,3-BDO production (FIG. 1A), rate (FIG. 1B), and yield [c-mol %] (FIG. 1C), independent from the oxygen transfer rate (OTR). Moreover, comparison of the valine levels relative to 1,3-BDO levels in multiple bacterial strains indicated that there was an inverse relationship between valine production and 1,3-BDO yield (FIG. 2A). Consistent with these results, the ilvGM deletion strains (L16410 and L16411) had negligible levels of valine (FIG. 2B), and increased levels of 1,3-BDO for each of the culture volumes (FIG. 2C). Specifically, the reduction of valine is translated into approximately 1.0-2.5 [c-mol %] increase in 1,3-BDO yield (FIG. 2C).

Collectively, these results demonstrated that attenuation of a valine biosynthesis enzyme decreased valine levels, which translated into an increase in 1,3-BDO yield.

### Example II

### Aldehyde Dehydrogenase with Specific Activity to Acetaldehyde and not R-3-Hydroxybutyraldehyde for Use in an Acetaldehyde Recycling Loop

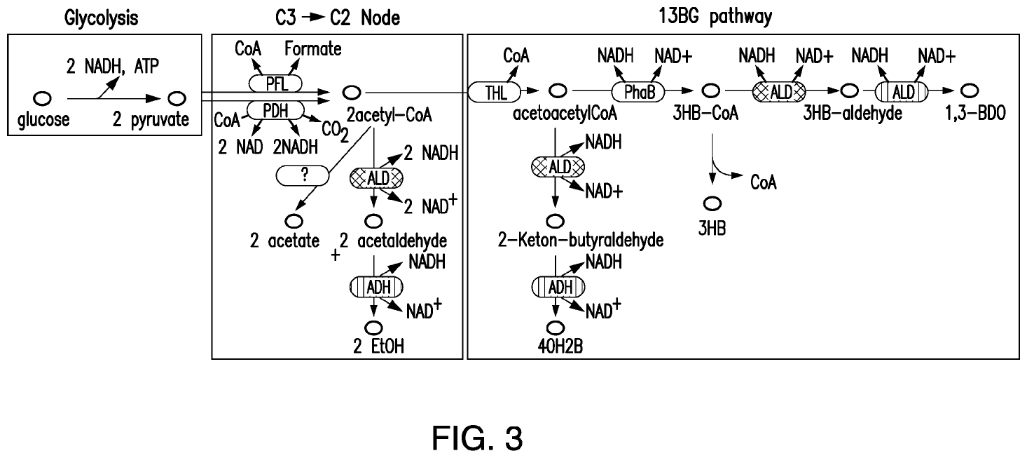

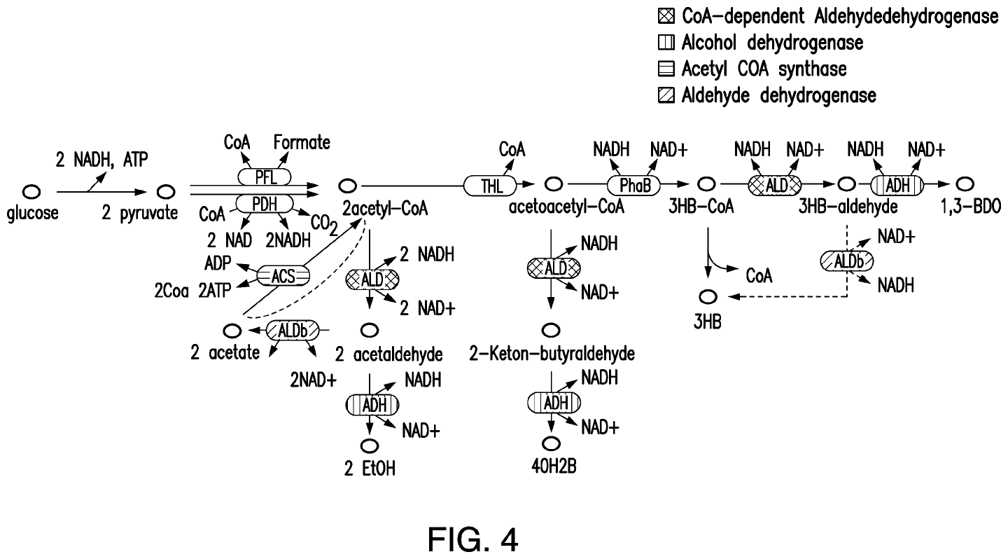

The following examples demonstrates that an aldehyde dehydrogenase enzyme with specificity for acetaldehyde can be used in an acetaldehyde recycling loop to reduce the levels of the acetyl-CoA by-products acetate and/or ethanol. As shown in FIG. 3, an exemplary pathway for the production of 1,3-BDO involves a thiolase (e.g., THL), an acetoacetyl-CoA reductase (e.g., PhaB), a CoA-dependent Aldehyde dehydrogenase (e.g., ALD), and an alcohol dehydrogenase (e.g., ADH). However, acetyl-CoA can also be converted to acetaldehyde by ALD, and acetaldehyde can be converted to ethanol by ADH, which limits the amount of acetyl-CoA that can be used for 1,3-BDO production. To reduce the ethanol and acetaldehyde by-products the inventors designed an exogenous recycling loop that could convert the acetaldehyde to acetate by an aldehyde dehydrogenase (e.g., AldB), and convert acetate back to acetyl-CoA by an acetyl-CoA synthase (e.g., ACS). However, as shown in FIG. 4, certain aldehyde dehydrogenase enzymes can act on 3HB-aldehyde, as well as acetaldehyde.

To identify an aldehyde dehydrogenase enzyme that is selective for acetaldehyde the lysate activities of selected AldB candidates were measured using three different aldehyde substrates: acetaldehyde (AcAld), and R-3-hydroxybutyraldehyde (R-3HBuAld) in order to gauge aldehyde selectivity.

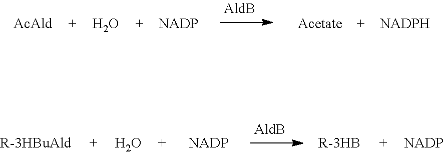

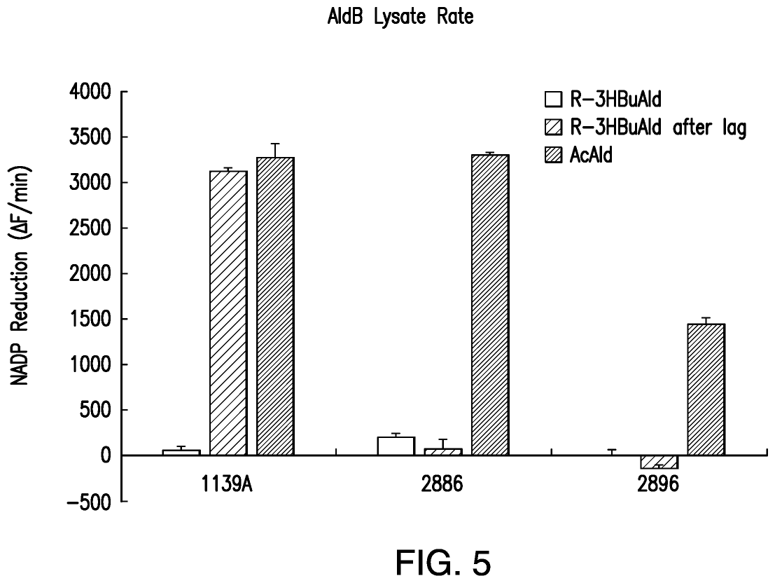

The results demonstrated that specific AldB enzymes could be identified that have specific activity for acetaldehyde and not 3-HB aldehyde (FIG. 5).

### Example III

### Inclusion of an Acetyl-CoA Synthase Variant in an Acetaldehyde Recycling Loop Increased 1,3-BDO Production

The following examples demonstrates that expression of an acetyl-CoA synthase variant in an acetaldehyde recycling loop increased 1,3-BDO production. The addition of the acetyl-CoA synthase variant (ACS*) was assayed in three different backgrounds of Ald-expressing strains: L16768 (Ald only), L16933 (Ald+pntAB and NadK), and L17786 (Ald with a NadK variant).

TABLE 2

| | Ald | ACS* | NadK* |
|---|---|---|---|
| L16768 | 714UC | | |
| L16946 | 714UC | X | |
| L16933 | 714AIX | | |
| L17662 | 714AIX | X | |
| L17786 | 714AIX | | X |
| L17787 | 714AIX | X | X |

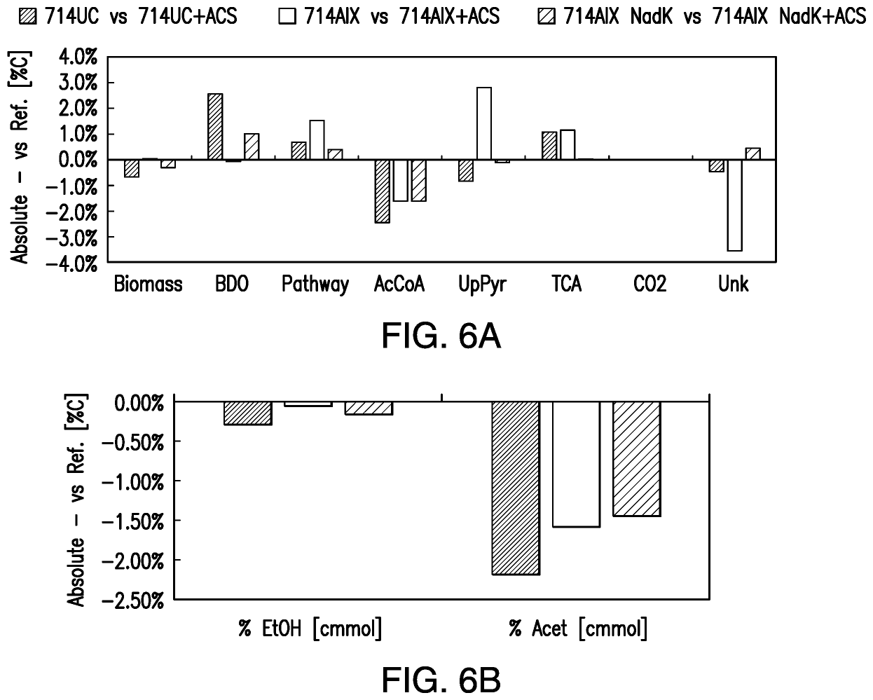

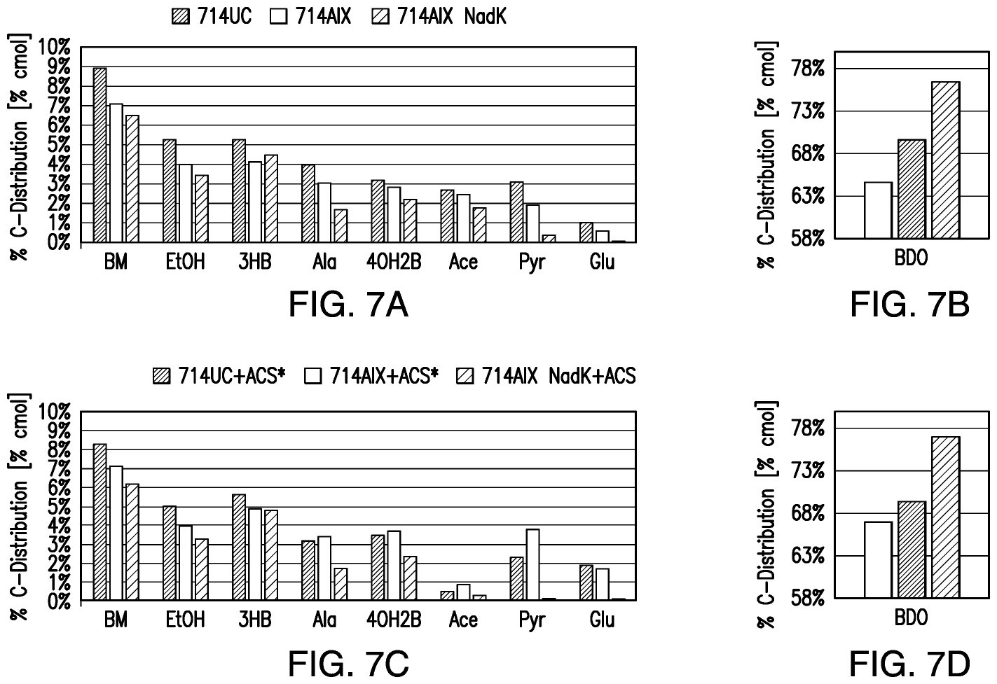

Comparison of the carbon distribution among pathway product and the carbon-3:carbon-2 node indicated that expression of ACS* improved the production of 1,3-BDO in the L16946 and L17787 strains, relative to the L16768 and L17787 strains, respectively (FIG. 6A). In addition, ACS* expression significantly reduced acetate, but not ethanol formation, indicating that the acetate recycle is efficiently competing with CoA hydrolase (FIG. 6B). Consistent with the carbon distribution, the overall product distribution demonstrated that expression of ACS* significantly reduced by-products (FIG. 7C) and increased 1,3-BDO production (FIG. 7D), relative to stains not expressing ACS* (FIG. 7A) and (FIG. 7B).

Taken together, these results demonstrate that exogenous expression of an acetyl-CoA synthase variant (ACS*) can decrease by-product production and increase 1,3-BDO production by specifically reducing acetate levels.

### Example IV

### Inclusion of an Acetyl-CoA Synthase Variant in an Acetaldehyde Recycling Loop Increased 1,3-BDO Production

The following examples demonstrates that co-expression of an acetyl-CoA synthase variant and an aldehyde dehydrogenase as part of an acetaldehyde recycling loop can decrease the by-product levels of acetate and ethanol, and increase 1,3-BDO production, in different strain backgrounds.

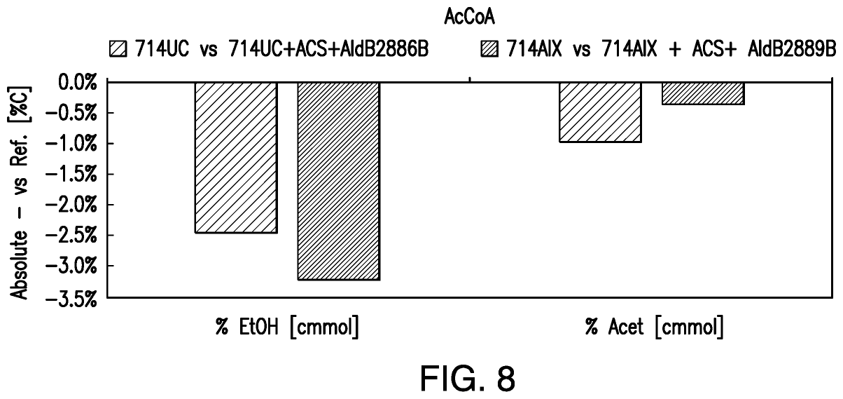

The effect of ACS* and AldB 2886B expression was tested in two different backgrounds of Ald-expressing strains. Comparison of the strains with or without co-expression of ACS* and AldB2886B demonstrated that ACS* and AldB expression significantly reduced both acetate and ethanol production (FIG. 8). Moreover, the overall carbon-2 (i.e., ethanol and acetate) reduction was similar between both strains.

TABLE 3

| | Ald | ACS* | AldB |
|---|---|---|---|
| L16768 | 714UC | | |
| L16948 | 714UC | X | p115-2886B |
| | | | (plasmid) |
| L16933 | 714AIX | | |
| L17663 | 714AIX | X | p108-2886B |
| | | | (plasmid) |

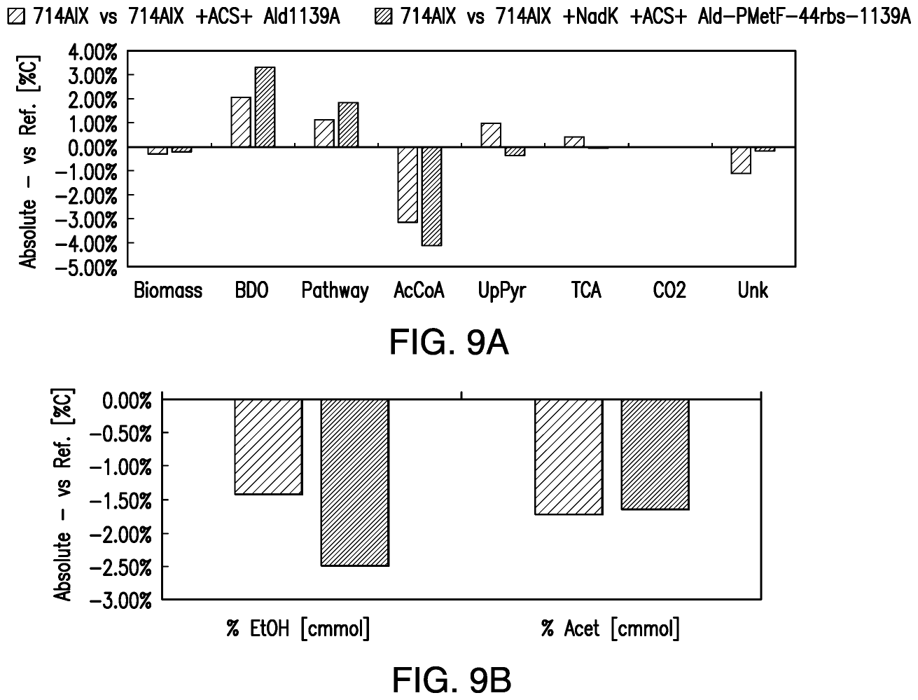

Similar results were achieved using a different AldB candidate, AldB 1139A (FIG. 9A and FIG. 9B). Comparison of strains overexpressing ACS* and the alternative AldB candidate (AldB 1139A), with or without the NadK variant (NadK*), resulted in an increase in 1,3-BDO (FIG. 9A) and a decrease in both ethanol and acetate (FIG. 9B), relative to their respective control strains without ACS* and AldB overexpression.

TABLE 4

| | Ald | ACS* | AldB | NadK* |
|---|---|---|---|---|
| L16933 | X | | | |
| L17674 | X | X | X | |
| L17786 | X | | | X |
| L17892 | X | X | X | X |

### Example V

### Inclusion of an Alanine-Recycling Loop Reduced Alanine

The following examples demonstrates that co-expression of a D-amino acid dehydrogenase (dadA) and an alanine racemase (dadX) as part of an alanine recycling loop decreased the by-product levels of alanine, and increased the levels of an exemplary acetyl-CoA derived product: 1,3-BDO.

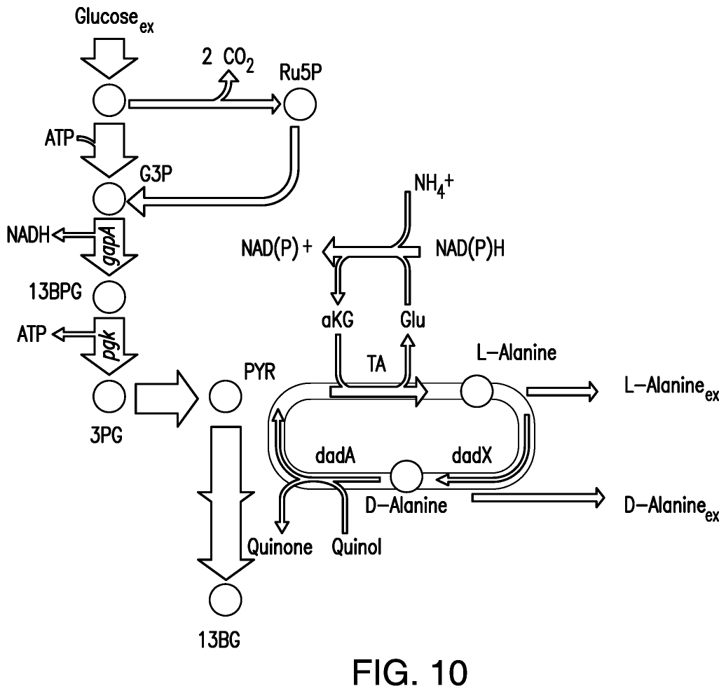

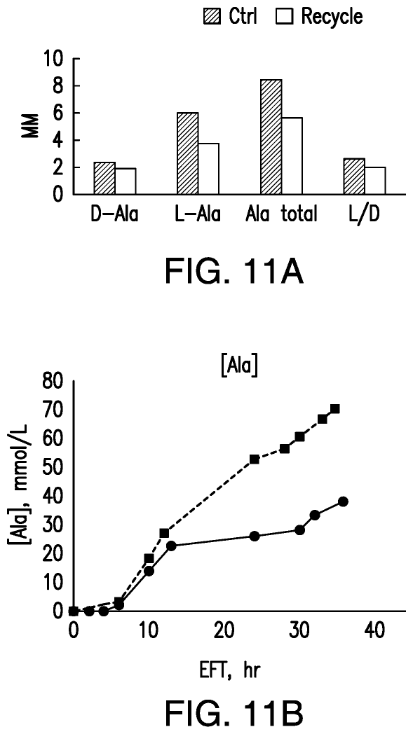

The carbon flux from pyruvate to acetyl-CoA can be limited when carbons are diverted from pyruvate to alanine metabolism. To test whether the unwanted alanine by-product could be recycled back to pyruvate, nucleic acids encoding D-amino acid dehydrogenase (dadA) and an alanine racemase (dadX) were over-expressed in E. coli thereby creating an alanine cycling loop (FIG. 10). Following fermentation, the alanine concentration between organisms with D-amino acid dehydrogenase (dadA) and alanine racemase (dadX), relative to control organisms without D-amino acid dehydrogenase (dadA) and alanine racemase (dadX) was compared. The results indicated that the overexpression of D-amino acid dehydrogenase (dadA) and alanine racemase (dadX) significantly reduced alanine concentration at the end of the fermentation process by approximately 43% (FIG. 11B, FIG. 11C, and Table 5). Therefore, the exogenous expression of D-amino acid dehydrogenase (dadA) and alanine racemase (dadX) can generate an alanine-recycling loop that can reduce the levels of alanine in the organism.

TABLE 5

| | D-Ala | L-Ala | Ala total | L-Ala/D-Ala Ratio |
|---|---|---|---|---|
| Control | 2.34 | 6.09 | 8.43 | 2.60 |
| dadA + dadX | 1.91 | 3.76 | 5.67 | 1.97 |

Analysis of the L- and D-forms of alanine indicated that the alanine-recycling loop reduces the L-alanine fraction to a greater extent than the D-alanine fraction (FIG. 11B and Table 5). Consistent with the reduced L-alanine fraction, the L-alanine/D-alanine ratio was also reduced in the organisms that expressed the alanine-recycling loop (FIG. 11B and Table 5).

Importantly, the reduced concentration of alanine in organisms that expressed the alanine recycling loop was detected across multiple aeration parameters without affecting other products, including pyruvate or 1,3 BG (FIG. 11A).

Taken together, these results demonstrated that the alanine-recycling loop, which involved co-expression of a D-amino acid dehydrogenase (dadA) and an alanine racemase (dadX), was able to reduce the unwanted by-product of alanine.

### Example VI

### Citrate Synthase Variant Decreased TCA Cycle Derived by-Products, and Increased 1,3-BDO

The following examples demonstrates that the expression of a citrate synthase variant decreased the unwanted tricarboxylic acid (TCA) cycle derived by-products, and increased the levels of an exemplary acetyl-CoA derived product: 1,3-BDO.

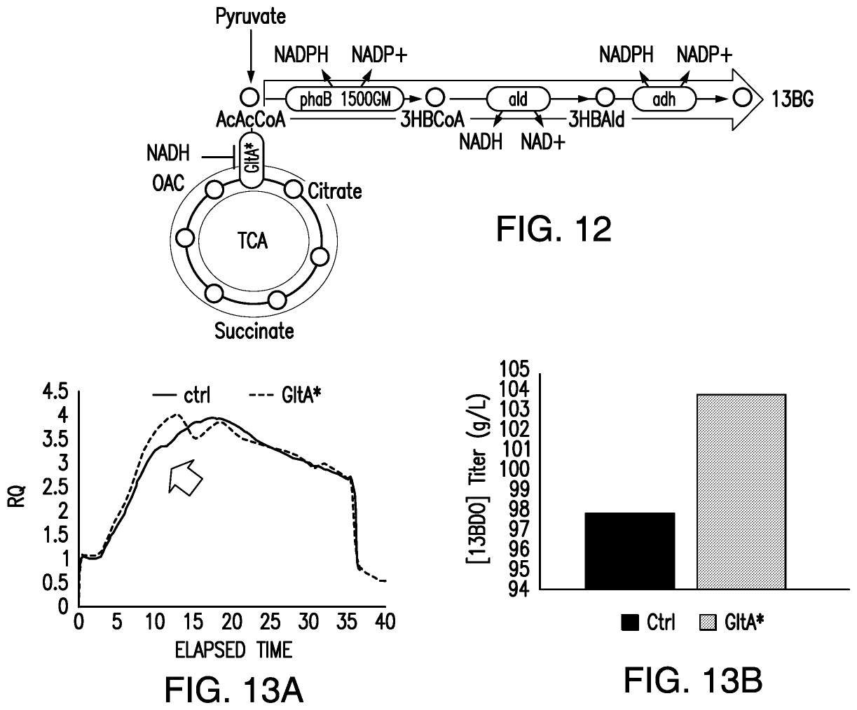

Citrate synthase is an enzyme that catalyzes the first step of the TCA by converting acetyl-CoA and oxaloacetate to form citrate, and is a key enzyme in pulling carbon flux away from acetyl-CoA towards other products such as, 1,3-BDO, fatty acid methyl esters, or other elongation chain products (FIG. 12). Consequently, citrate synthase produces unwanted TCA cycle derived by-products.

Citrate synthase can be strongly and specifically inhibited by NADH, and it has been reported that a citrate synthase variant (gltA R109L) has increased sensitivity to NADH (see Stokell et al., J. Biol. Chem, 2003; 278(37):35435-35443). Therefore, to test whether inhibition of citrate synthase could decrease unwanted tricarboxylic acid (TCA) cycle derived by-products, and increase 1,3-BDO production, exogenous gltA R109L was expressed in E. coli.

Measurement of the exemplary acetyl-CoA derived product, 1,3-BDO, following the over-expression of the citrate synthase variant (gltA R109L) revealed that the citrate synthase variant increased 1,3-BDO titer (FIG. 13B). In addition, lower TCA cycle derived by-products were produced in the microorganisms expressing the citrate synthase variant (gltA R109L). Notably, the microorganisms expressing the citrate synthase variant (gltA R109L) also had a fast microaerobic transition (FIG. 13A).

Taken together, these results demonstrate that a citrate synthase variant is able to reduce the production of unwanted TCA cycle derived by-products, and increase the titer of acetyl-CoA derived products, such as 1,3-BDO.

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1. A non-naturally occurring microbial organism having reduced by-products, comprising a microbial organism having a glycolysis pathway and an enhanced carbon flux through acetyl-CoA, the microbial organism comprising one or more of:
(a) an attenuated acetolactate synthase;
(b) an acetaldehyde recycling loop;
(c) an alanine recycling loop; and
(d) a citrate synthase variant.

2. The non-naturally occurring microbial organism of claim 1, wherein the attenuated acetolactate synthase comprises: (a) reduced expression of acetolactate synthase; (b) a deletion of acetolactate synthase; or (c) a non-functional acetolactate synthase.

3.-4. (canceled)

5. The non-naturally occurring microbial organism of claim 1, wherein: (a) the acetolactate synthase is ilvG; (b) the attenuated acetolactate synthase decreases the biosynthesis of valine, as compared to a microbial organism having a wild-type acetolactate synthase; or (c) a combination of (a) and (b).

6. (canceled)

7. The non-naturally occurring microbial organism of claim 1, wherein the acetaldehyde recycling loop comprises at least one exogenous nucleic acid encoding an acetaldehyde recycling loop enzyme selected from the group consisting of an aldehyde dehydrogenase and an acetyl-CoA synthase.

8. The non-naturally occurring microbial organism of claim 7, wherein the aldehyde dehydrogenase is encoded by aldB, or wherein the acetyl-CoA synthase is an acetyl-CoA synthase variant.

9. (canceled)

10. The non-naturally occurring microbial organism of claim 7, wherein said at least one exogenous nucleic acid is a heterologous nucleic acid.

11. The non-naturally occurring microbial organism of claim 1, wherein the non-naturally occurring microbial organism has reduced acetate, ethanol, or a combination thereof.

12. The non-naturally occurring microbial organism of claim 1, wherein the alanine recycling loop comprises at least one exogenous nucleic acid encoding an alanine recycling loop enzyme selected from the group consisting of a D-amino acid dehydrogenase and an alanine racemase.

13-14. (canceled)

15. The non-naturally occurring microbial organism claim 7, wherein:
(a) the alanine-recycling loop comprises at least one exogenous nucleic acid encoding a D-amino acid dehydrogenase and an alanine racemase;
(b) the D-amino acid dehydrogenase is encoded by dadA and/or the alanine racemase is encoded by dadX;
(c) the non-naturally occurring microbial organism has a reduced alanine concentration, as compared to a microbial organism without an alanine recycling loop, wherein optionally the reduced alanine concentration is a reduced L-alanine concentration; or
(d) a combination of (a) to (c).

16. The non-naturally occurring microbial organism of claim 12, wherein said at least one exogenous nucleic acid is a heterologous nucleic acid.

17-20. (canceled)

21. The non-naturally occurring microbial organism of any one of claim 1, wherein the non-naturally occurring microbial organism has reduced a ratio of L-alanine to D-alanine, as compared to a microbial organism without an alanine recycling loop.

22. The non-naturally occurring microbial organism of claim 1, wherein the citrate synthase variant is a Type II citrate synthase.

23. The non-naturally occurring microbial organism of claim 22, wherein:
(a) the citrate synthase variant binds NADH with greater affinity than wild-type citrate synthase;
(b) the citrate synthase variant comprises at least one exogenous nucleic acid encoded by gltA R109L;
(c) the non-naturally occurring microbial organism has reduced tricarboxylic acid cycle (TCA) cycle derived by-products, as compared to a microbial organism with a wild-type citrate synthase; or
(d) a combination of (a) to (c).

24. (canceled)

25. The non-naturally occurring microbial organism of claim 23, wherein said at least one exogenous nucleic acid is a heterologous nucleic acid.

26. (canceled)

27. The non-naturally occurring microbial organism of claim 1, wherein the non-naturally occurring microbial organism is in a substantially anaerobic culture medium.

28. The non-naturally occurring microbial organism of claim 1, further comprising an 1,3-butanediol (1,3-BDO) pathway, an (3R)-hydroxybutyl (3R)-hydroxybutyrate pathway, a 3-hydroxybutyryl-coenzyme A (3HB-CoA) pathway, a methyl methacrylate (MMA) pathway, an adipate pathway, a caprolactam pathway, a 6-aminocaproic acid (6-ACA) pathway, a hexametheylenediamine (HMDA) pathway, or a methacrylic acid (MAA) pathway.

29-47. (canceled)

48. The non-naturally occurring microbial organism of claim 1, wherein the microbial organism is a species of bacteria, yeast, or fungus.

49. A method for enhancing the carbon flux through acetyl-CoA in a non-naturally occurring microbial organism to increase the yield of an acetyl-CoA derived product, the method comprising culturing the non-naturally occurring microbial organism of claim 1 under conditions and for a sufficient period of time to produce the acetyl-CoA derived product.

50. The method of claim 49, wherein the acetyl-CoA derived product is selected from a group consisting of 1,3-butanediol (1,3-BDO), methyl methacrylate (MMA), 3R-hydroxybutyric acid-3R-hydroxybutryrate, 3-hydroxybutyrate (3-HB), 4-hydroxy-2-butanone (4OH2B), hexamethylenediamine (HMDA), caprolactam, adipate, 6-aminocaproic acid (6-ACA), and methacrylic acid (MAA).

51-53. (canceled)
